(19) United States
(12) Patent Application Publication
(10) Pub. No.: US 2018/0099155 A1
(43) Pub. Date: Apr. 12, 2018

(54) APPARATUS AND METHOD FOR VISUALIZING A HADRON BEAM PATH TRAVERSING A TARGET TISSUE BY MAGNETIC RESONANCE IMAGING

(71) Applicant: Ion Beam Applications S.A., Louvain-la-Neuve (BE)

(72) Inventors: Damien PRIEELS, Court-Saint-Etienne (BE); Erik VAN DER KRAAIJ, Rixensart (BE); Sébastien HENROTIN, Watermael-Boitsfort (BE); Caterina BRUSASCO, Bossière (BE)

(73) Assignee: Ion Beam Applications S.A.

(21) Appl. No.: 15/727,292

(22) Filed: Oct. 6, 2017

(30) Foreign Application Priority Data
Oct. 7, 2016 (EP) 16192732.2

(51) Int. Cl. A61N 5/10; G01R 33/48; G01R 33/483; G01R 33/56; A61B 5/055; A61B 5/00

(52) U.S. Cl. CPC A61N 5/1039; G01R 33/4808; G01R 33/4833; G01R 33/5608; A61N 2005/1074; A61B 5/743; A61N 2005/1085; A61N 2005/1055; A61B 5/055

(57) ABSTRACT

The present disclosure relates to a method and a medical apparatus for visualizing on magnetic resonance (MR) images a hadron beam path traversing an organic body. The present method may utilize artefacts in MR image acquisition provoked by the changes in properties of excitable atoms when irradiated by a hadron beam. By synchronizing the hadron pulses with different steps of MR data acquisition, it is possible to identify such artefacts and determine, based on their positions, the hadron beam path and the corresponding position of the Bragg peak.

### CROSS-REFERENCE TO RELATED APPLICATIONS

This application claims the benefit of priority to European Application No. 1619272.2, filed Oct. 7, 2016, the contents of which are incorporated herein by reference.

### TECHNICAL FIELD

The present disclosure relates to a medical apparatus comprising a charged hadron therapy device coupled to a magnetic resonance imaging device (MRI) adapted for visualizing in situ a hadron beam traversing a target tissue relative such that the position of the Bragg peak relative to the position of a target spot in said target tissue may be assessed. The in situ localization of the actual position of the Bragg peak relative to the target spot, immediately before a hadron therapy session starts, may be highly useful for validating the planned position of the Bragg peak of the hadron beam determined during an earlier established treatment plan for treating said target spot. If a discrepancy appears between the planned and actual positions of the Bragg peak of the hadron beam, embodiments of the present disclosure may allow the correction of the initial energy, E1, of the hadron beam required for positioning the Bragg peak over the target spot. Accordingly, the hadron therapy session may not need to be cancelled and instead proceed with corrected parameters.

### BACKGROUND

Hadron therapy (for example, proton therapy) for treating a patient may provide several advantages over conventional radiotherapy. These advantages are generally due to the physical nature of hadrons. For example, a photon beam in conventional radiotherapy releases its energy according to a decreasing exponential curve as a function of the distance of tissue traversed by the photon beam. By contrast, and as illustrated in the example of FIG. 2A, a hadron beam first releases a small fraction of its energy as it penetrates tissues 41-43, forming a plateau, then, as the hadron path is prolonged, releases energy locally following a steep increase to a peak and a fall-off at the end of the range of the beam. The peak is called a Bragg peak and corresponds to the maximum of the Bragg curve illustrated in the example of FIG. 2C. Consequently, a hadron beam may deliver a high dose of hadrons at a precise location within a target tissue 40 and may therefore preserve the surrounding healthy tissues 41-44. As illustrated in the example of FIG. 2A, if the position, BP0, of the Bragg peak of a hadron beam is offset relative to the target tissues 40, high doses of hadrons may be delivered to adjacent tissues 43, 44, which are healthy (as illustrated with solid line, E0, and dashed line, E0d,of the curves of energy loss, Eₗₒₛₛ, with respect to the distance, Xh, travelled by the hadron beam within tissues and measured along the beam path, Xp, in the example of FIG. 2A). For this reason, the determination of the relative position of the Bragg peak with respect to the position of the target tissue is often crucial to properly implement hadron therapy to a patient.

In practice, hadron therapy usually requires the establishment of a treatment plan before any treatment can start. During this treatment plan, a computer tomography scan (CT scan) of the patient and target tissues is generally performed. The CT scan may be used to characterize the target tissue 40 and the surrounding tissues 41-43 to be traversed by a treatment hadron beam 1h for the treatment of a patient. The characterization may yield a 3D representation of the volume comprising the target tissue, and a treatment plan system may determine a range-dose calculated based on the nature of the tissues 41-43 traversed by the hadron beam.

This characterization may allow computation of a water equivalent path length (WEPL), which may be used for determining the initial energy, Ek, of the treatment hadron beam required for delivering a prescribed dose of hadrons to a target spot 40s, wherein k=0 or 1, depending on the stage when said initial energy was determined. The example of FIG. 2C illustrates the conversion of the physical distances travelled by a hadron beam traversing different tissues into corresponding WEPLs. The WEPL of a hadron beam travelling a given distance through a given tissue is the equivalent distance said hadron beam would travel in water. As illustrated in the example of FIG. 2C if, as is often the case, healthy tissues 41-43 of different natures and thicknesses separate a target tissue from the outer surface of the skin of a patient, the WEPL of a target spot may be calculated taking into account the water corresponding path lengths of each tissue in series until the target spot is reached. With a value of the equivalent path length of a hadron beam traveling in water, the initial energy, Ek, required for positioning the Bragg peak at the WEPL of the target spot may be computed and correspond to the initial energy, Ek, required for positioning the Bragg peak at the target spot within the target tissue.

The treatment plan may then be executed during a treatment phase including one or more treatment sessions during which doses of hadrons are deposited onto the target tissue. The position of the Bragg peak of a hadron beam with respect to the target spots of a target tissue, however, may suffer of a number of uncertainties including:
the variations of the patient position, on the one hand, during a hadron therapy session and, on the other hand, between the establishment of the treatment plan and the hadron therapy session;
the variations of the size and/or of the position of the target tissue (see, for example, FIG. 2B) and/or of the healthy tissues 41-43 positioned upstream from the target tissue with respect to the hadron beam; and
the range calculation from CT scans being limited by the quality of the CT images. Another limitation is linked to the fact that CT scans use the attenuation of X-rays that have to be converted in hadron attenuation, which may depend on the chemical composition of the tissues traversed.

The uncertainty on the position of the patient and, in particular, of the target tissue may be critical. Even with an accurate characterization by CT scan, the actual position of a target tissue during a treatment session may remain difficult to ascertain for the following reasons:
(A) first, during an irradiation session, the position of a target tissue may change because of anatomical processes such as breathing, digestion, or heartbeats of the patient. Anatomical processes may also cause gases or fluids appearing or disappearing from the beam path, Xp, of a hadron beam.
(B) second, treatment plans are generally determined several days or weeks before a hadron treatment session starts and treatment of a patient may take several weeks distributed over several treatment sessions. During this time period, the patient may lose or gain weight, therefore modifying, sometimes significantly, the volume of tissues such as fats and muscles.

Accordingly, the size of the target tissue may change (e.g., a tumour may have grown, receded, or changed position or geometry). The example of FIG. 2B shows an example of evolution of the size and position of a target tissue 40 between the time, t0, of the establishment of the treatment plan and the times, t0+Δt1, t0+Δt2, t1=t0+Δt3, of treatment sessions. The treatment plan and last treatment session may be separated by several days or weeks. The treatment plan established at time, t0, may therefore comprise irradiation of a target spot 40sij (black spot in the example of FIG. 2B), which belonged to the target tissue 40p at said time, t0. Because the target tissue 40p may have moved or changed shape during the time period, Δt3, said target spot 40si,j may not belong to the target tissue 40 anymore at the time, t0+Δt3, of the treatment session and may be located in a healthy tissue instead. Consequently, irradiating said target spot may hit and possibly harm healthy tissues 43 instead of target tissues 40.

The use of a magnetic resonance imaging device (MRI) coupled to a hadron therapy device has been proposed for identifying any variation of the size and/or the position of a target tissue. For example, U.S. Pat. No. 8,427,148 generally relates to a system comprising a hadron therapy device coupled to an MRI. Said system may acquire images of the patient during a hadron therapy session and may compare these images with CT scan images of the treatment plan. FIG. 1 illustrates an example of a flowchart of a hadron therapy session using a hadron therapy device coupled to an MRI. A treatment plan may be established including the characterization of the target tissue 40s and surrounding tissues 41-43. This step is generally performed with a CT scan analysis and allows the determination of the position, P0, and morphology of a target tissue, the best trajectories or beam paths, Xp, of hadron beams for the hadron treatment of the target tissue, and characterization of the sizes and natures of the tissues traversed by a hadron beam following said beam paths, Xp, to determine WEPLs of target spots of the said target tissue. The initial energies, Ek, of the hadron beams required for matching the corresponding positions, BP0, of the Bragg peaks of the hadron beams to the position, P0, of the target tissue may thus be calculated. This generally completes the establishment of a treatment plan.

A hadron therapy session may follow the establishment of the treatment plan. With an MRI coupled to a hadron therapy device, it may be possible to capture a magnetic resonance (MR) image of a volume, Vp, including the target tissue and surrounding tissues to be traversed by a hadron beam. The MR image may then be compared with CT scan images to assess whether any morphological differences, A, can be detected in the imaged tissues between the time the CT scans were performed (=t0 in the example of FIG. 2B) and the time of the hadron therapy session (t1=t0+Δt3 in the example of FIG. 2B). If no substantial difference in morphology affecting the treatment session is detected, then the hadron therapy session may proceed as planned in the treatment plan. If, on the other hand, some differences are detected that could influence the relative position of the target tissue with respect to the planned hadron beams and their respective Bragg peaks, the hadron therapy session may be interrupted and a new treatment plan established. This technique may prevent carrying out a hadron therapy session based on a treatment plan that has become obsolete, which may prevent healthy tissues from being irradiated instead of the target tissue.

The magnetic resonance (MR) images generally provide high contrast of soft tissue traversed by a hadron beam but, at the time of filing, have usually not been suitable for visualizing the hadron beam itself, let alone the position of the Bragg peak because:
MRI measures the density of hydrogen atoms in tissues but, at the time of filing, does not usually yield any identifiable information on the hadron stopping power ratio. The conversion from density of hydrogen atoms to the hadron stopping power ratio suffers from uncertainties similar to and yet generally less understood than those of the conversion from X-rays in CT scan.
Due to the different techniques used in CT scan and in MRI, the comparison between the images from CT scan and the images from MRI may suffer from uncertainties.

In conclusion, in hadron therapy, an accurate determination of the position of the Bragg peak relative to the portion of a target tissue is important because errors regarding this position may lead to the irradiation of healthy tissues rather than irradiation of target tissues.

However, no satisfactory solution for determining the relative positions of the Bragg peak and target tissues is presently available. Apparatuses combining a hadron therapy device and an MRI may allow in situ acquisition of images during a treatment session, thus giving information related to the actual position of the target tissue.

For example, EP Pat. application No. 2196241 generally relates to a therapeutic apparatus comprising a vertical field MRI scanner in combination with a fixed charged particle guiding means, entering through an opening at the top of the magnet. This arrangement may reduce the curvature of charged particle paths due to the magnetic field of the MRI magnet. The charged particle beam may be oriented at an angle of approximately 20 degrees relative to the vertical axis of the magnet. This may allow the application of multiple field treatment by rotating the subject support about the vertical axis, without a complicated rotation system on the charged particle beam line.

PCT application No. WO2009156896 generally relates to a radiation therapy system comprising: (a) a radiation therapy subsystem configured to apply radiation pulses to a region of a subject at pulse intervals (Tpi); (b) a magnetic resonance (MR) imaging subsystem configured to acquire a dataset of MR imaging data samples from said region over MR sampling intervals, TAQ, overlapping at least some of the pulse intervals and being longer than the pulse intervals, Tpi, (TAQ>Tpi); (c) a synchronizer configured to identify MR overlapping imaging data samples defined as MR imaging data samples of the dataset whose acquisition times overlap pulse intervals; and (d) a reconstruction processor configured to reconstruct the dataset without the MR overlapping imaging data samples to generate a reconstructed MR image.

The images generated by the foregoing systems are, however, generally insufficient for ensuring a precise determination of the position of the Bragg peak of a hadron beam and of its location relative to the target tissue. Accordingly, there remains a need for a hadron therapy device combined with an MRI that allows a better determination of the position of the Bragg peak relative to the position of a target tissue.

### SUMMARY

In one embodiment according to the present disclosure, a method for visualizing a hadron beam traversing an organic body may comprise:
(a) providing a hadron source adapted for directing a hadron beam having an initial energy, E0, along a beam path intersecting a target tissue in the organic body;
(b) providing a magnetic resonance imaging device (MRI) for acquiring magnetic resonance data within an imaging volume, Vp, including the target tissue, positioned in a uniform main magnetic field, B0;
(c) acquiring magnetic resonance data from the imaging volume, by applying at least the following MR-data acquisition steps:
   a layer selection step (MRv) for selecting an imaging layer, Vpi, of the imaging volume, Vp, of thickness, Δxi, measured along the first direction, X1 including creating a magnetic field gradient in a first direction, X1;
   an excitation step (MRe) for exciting the spin of the nuclei of excitable atoms A0, by creating an electromagnetic field, B1, oscillating at a given RF frequency range [fL]i corresponding to the Larmor frequencies of the excitable atoms located within the imaging layer, Vpi, during an excitation period, Pe=(te1−te0), wherein te0 and te1 are the times of the beginning and end of the excitation step, respectively;
   a phase gradient step (MRp) for localising along the second direction, X2, the origin of RF signals received by the antennas during relaxation of the excited spins, including creating magnetic field gradients in a second direction, X2, normal to the first direction, X1, (X1 ⊥X2), during a period Pp=(tp1−tp0), wherein tp0 and tp1 are the times of the beginning and end of the phase gradient step, respectively, with tf0>tp1; and
   a frequency gradient step (MRf) for localising along the third direction, X3, the origin of RF signals received by the antennas during relaxation of the excited spins including creating magnetic field gradients in a third direction, X3, normal to the first and second directions, (X1⊥X2⊥X3), during a period Pf =(tf1−tf0), wherein tf0 and tf1 are the times of the beginning and end of the frequency gradient step, respectively, with tf0>tp1;
(d) directing a hadron beam having the initial energy, E0, along a beam path intersecting said target body in the imaging layer, Vpi, in a number, N, of hadron pulses of pulse periods, PBi, wherein, N is an integer greater than 0;
(e) representing on a display the organic body from the magnetic resonance data acquired by the MRI within the imaging volume, Vp, and
(f) on the same display, visualizing the beam path in the target tissue as a hyposignal, weaker than the signal generated by the excitable atoms which are not exposed significantly to the effects of the hadron beam.

In some embodiments, the acquisition of magnetic resonance data and the emission of hadron pulses may be synchronized such that an MR-period, Pj, with j=e, f, and/or p, of one or more of the excitation step, Pe, the phase gradient step, Pp, and the frequency gradient step, Pf, overlaps with and does not exceed the pulse period, PBi, by more than 10%, Pj≤1.1 PBi, and in that, the MR-period, Pj, is out of phase with respect to the pulse period PBi of each of the overlapping hadron pulses by not more than 10%, such that (tBi,0−tj0)/PBi≤0.1, and (tBi,1−tj1)/PBi≥−0.1, wherein tBi,0 and tBi,1 are the times of the beginning and end of each hadron pulse, and wherein j=e, f, and/or p.

In one embodiment, the MR-period, Pj, may be the excitation period, Pe, which ends close to the end of the pulse period, PBi, such that (tBi,1−te1)/PBi≤0, and such that (tBi,1−te1)/PBi≤0.3, e.g., ≤0.2 or ≤0.1, or (tBi,1−te1)/PBi=0.

The N hadron pulses may have a period, PBi, between 10 μs and 30 ms. Depending on the type of hadron source used, PBi may be between 1 and 10 ms or, alternatively, between 5 and 20 ms. Two consecutive hadron pulses may be separated from one another by a period, APBi, between 1 and 20 ms. Each of the excitation period, Pe, phase gradient period, Pp, frequency gradient period, Pf, may be independently from one another selected between 1 and 100 ms, e.g., between 5 and 50 ms.

The MR-data acquisition steps may further comprise additional sequences as defined above, comprising one or more of a layer selection step simultaneously with an excitation step, of a phase gradient step and/or of a frequency gradient step, in different orders and periods, Pj. These additional MR-data acquisition steps may or may not be synchronized with the emissions of hadron pulses as defined above. In some embodiments, the hadron pulses may be synchronized with as many data acquisition steps as possible, which may strengthen the signal representative of the beam path.

In order to capture a trace of the beam path over its entire path length, the beam path of the hadron beam may be substantially normal to the first direction, X1. The imaging volume, Vp, may be controlled by creating a magnetic gradient along, one, two, or three of the first, second, and third directions, X1, X2, X3. Accordingly, a thickness of the imaging volume along said first, second, or third directions, X1, X2, X3 may be controlled.

A treatment session may be planned in two steps: first at time, t0, leading to the 2 5 establishment of a treatment plan, and second at a time, t1>t0, when a therapy session is to take place, and during which it may be assessed whether the validity of the results established in the treatment plan are still applicable at time, t1. In particular, the method may comprise:
(a) establishing at day, t0, a treatment plan and determining an initial energy, E0, of a hadron beam for depositing a given dose of hadrons to a target spot,
(b) comparing on the display the morphology and thicknesses of the tissues traversed by a hadron beam of initial energy, E0, at day, t1>t0, with the morphology and thicknesses of the same tissues as defined in the treatment plan, at day, t0,
(c) visualizing on the same display the actual position of the Bragg peak of the hadron beam, and
(d) in case of mismatch between the actual position of the Bragg peak and of the target tissue 40s, correcting the initial energy, E1, of the hadron beam required for the Bragg peak to fall over the target spot.

The imaging volume, Vp, may be controlled by creating a magnetic gradient along, one, two, or three of the first, second, and third directions, X1, X2, X3. Accordingly, a thickness of the imaging volume along said first, second, or third directions, X1, X2, X3 may be controlled.

It may be desired to create an MR image of the target tissue devoid of any artefacts caused by the irradiation of the excitable atoms by a hadron beam. Accordingly, embodiments of the present disclosure also include a method for visualizing an organic body traversed by a hadron beam without artefacts created by said hadron beam. The method may comprise:
(a) providing a hadron source adapted for directing a hadron beam having an initial energy, E0, along a beam path, Xp, intersecting a target tissue in the organic body;
(b) providing a magnetic resonance imaging device (MRI) for acquiring magnetic resonance data within an imaging volume, Vp, including the target tissue, positioned in a uniform main magnetic field, B0;
(c) acquiring magnetic resonance data from the imaging volume, by applying at least the following steps:
   a layer selection step (MRv) for selecting an imaging layer, Vpi, of the imaging volume, Vp, of thickness, Δxi, measured along the first direction, X1 including creating a magnetic field gradient in a first direction, X1;
   an excitation step (MRe) for exciting the spin of the nuclei of excitable atoms A0, by creating an oscillating electromagnetic field, B1, at a given RF frequency range [fL]i corresponding to the Larmor frequencies of the excitable atoms located within the imaging layer, Vpi, during an excitation period, Pe=(te1-31 te0), wherein te0 and te1 are the times of the beginning and end of the excitation step, respectively;
   a phase gradient step (MRp) for localising along the second direction, X2, the origin of RF signals received by the antennas during relaxation of the excited spins, including creating magnetic field gradients in a second direction, X2, normal to the first direction, X1, (X1⊥X2), during a period Pp=tp1-31 tp0), wherein tp0 and tp1 are the times of the beginning and end of the phase gradient step, respectively, with tf0>tp1; and
   a frequency gradient step (MRf)) for localising along the third direction, X3, the origin of RF signals received by the antennas during relaxation of the excited spins including creating magnetic field gradients in a third direction,
   X3, normal to the first and second directions, (X1⊥X2⊥X3), during a period Pf=(tf1-31 tf0), wherein tf0 and tf1 are the times of the beginning and end of the frequency gradient step, respectively, with tf0>tp1;
(d) directing a hadron beam having the initial energy, E0, along a beam path intersecting said target body in the imaging layer, Vpi, preferably normal to the first direction, X1, in a number, N, of hadron pulses of pulse periods, PBi, wherein, N is an integer greater than 0; and
(e) representing on a display the organic body from the magnetic resonance data acquired by the MRI within the imaging volume, Vp, without interferences from the hadron beam,
   characterized in that, the acquisition of magnetic resonance data and the emission of hadron pulses are synchronized such that
a pulse period PBi, overlaps with an MR-period, Pj, with j=e, f, and/or p, of one of the excitation step, Pe, the phase gradient step, Pp, and the frequency gradient step, Pf, and is not more than 20% of the MR-period, Pj, it overlaps with, such that PBi≤0.2 Pj, or
a pulse period PBi, does not overlap with any of the MR-periods, Pj.

The present disclosure also includes a medical apparatus, which may comprise:
(a) a hadron source adapted for directing a hadron beam having a beam energy, E1, along a beam path in a number, N, of hadron pulses of pulse period, PBi=(tBi,1−tBi,0), wherein, N is an integer greater than 0, tBi,0 is the time of the beginning of the i^{th} hadron pulse, and tBi,1 is the end of the i^{th} hadron pulse, said beam path intersecting an organic body containing excitable atoms (in particular hydrogen);
(b) a magnetic resonance imaging device (MRI) for the acquisition of magnetic resonance data from the excitable atoms within an imaging volume, Vp, including the organic body, wherein the MRI comprises:
   a main magnetic unit for creating a uniform main magnetic field, B0;
   an RF unit, suitable for creating an oscillating electromagnetic field, B1, at a given RF frequency range;
   slice selection coils for creating a magnetic field gradient in a first direction, X1;
   X2-gradient coils for creating magnetic field gradients in a second direction, X2, normal to the first direction, X1, (X1⊥X2);
   X3-gradient coils for creating magnetic field gradients in a third direction, X3, normal to the first and second directions, (X1⊥X2⊥X3);
   antennas for receiving RF signals emitted by excited atoms upon relaxation;
(c) a controller configured for acquiring magnetic resonance data by implementing the following steps:
   an excitation step (MRe) for exciting the spin of the nuclei of the excitable atoms, during an excitation period, Pe=(te1-31 te0), wherein te0 and te1 are the times of the beginning and end of the excitation step, respectively;
   a layer selection step (MRv) applied during the excitation step for selecting a layer, Vpi, of the imaging volume, Vp, of thickness, Δxi, measured along the first direction, X1;
   a phase gradient step (MRp) applied after the excitation and slice selection steps for localising along the second direction, X2, the origin of RF signals received by the antennas, during a period, Pp=(tp1−tp0), wherein tp0 and tp1 are the times of the beginning and end of the phase gradient step, respectively, with tp0>te1; and
   a frequency gradient step (MRf) applied after the phase gradient step for 3 0 localising along the third direction, X3, the origin of RF signals received by the antennas, during a period Pf=(tf1−tf0), wherein tf0 and tf1 are the times of the beginning and end of the frequency gradient step, respectively, with tf0>tp1; and
(d) a display for representing the organic body from the magnetic resonance data acquired by the MRI within the imaging volume, Vp, as well as for visualizing the beam path,
   characterized in that, the controller is further configured for synchronizing the acquisition of magnetic resonance data and the emission of hadron pulses, such that an MR-period, Pj, with j=e, f, and/or p, of one or more of the excitation step, Pe, the phase gradient step, Pp, and the frequency gradient step, Pf, overlaps with and does not exceed the pulse period, PBi, by more than 10%, Pj≤1.1 PBi, and in that, the MR-period, Pj, is out of phase with respect to the pulse period PBi by not more than 10%, such that (tBi,0−tj0)/PBi≤0.1, and (tBi,1−tj1)/PBi≥−0.1, wherein tBi,0 and tBi,1 are the times of the beginning and end of each hadron pulse, and wherein j=e, f, and/or p.

### BRIEF DESCRIPTION OF THE DRAWINGS

These and further aspects of the present disclosure will be explained in greater detail by way of example and with reference to the accompanying drawings in which:

FIG. 1 shows a flowchart of a hadron therapy method using a hadron therapy device coupled to a MRI.

FIG. 2A schematically shows the position of the Bragg peak of a hadron beam traversing tissues.

FIG. 2B schematically shows changes with time of the morphology and position of a target tissue that can create a discrepancy between a treatment plan and an actual required treatment.

FIG. 2C schematically shows the relationship between actual path lengths and water equivalent path lengths.

FIG. 3A schematically shows a medical apparatus comprising a hadron therapy device coupled to an MRI, according to an example embodiment of the present disclosure.

FIG. 3B schematically shows another medical apparatus comprising a hadron therapy device coupled to an MRI, according to another example embodiment of the present disclosure.

FIG. 4A schematically illustrates a nozzle mounted on a gantry for delivering a therapeutic dose of hadron, according to an example embodiment of the present disclosure.

FIG. 4B illustrates volumes of target tissue receiving a therapeutic dose of hadron from the nozzle of FIG. 4A, according to an example embodiment of the present disclosure.

FIG. 4C illustrates a dose of hadron delivered to the target tissue of FIG. 4B, according to an example embodiment of the present disclosure.

FIG. 5A schematically shows a selection of an imaging slice in an MRI, according to an example embodiment of the present disclosure.

FIG. 5B schematically shows a creation of phase gradients and frequency gradients during imaging of the slice of FIG. 5A, according to an example embodiment of the present disclosure.

FIG. 6A shows an example of an apparatus according to an example embodiment of the present disclosure, showing access of a hadron beam to a target tissue.

FIG. 6B shows another example of an apparatus according to another example embodiment of the present disclosure, showing access of a hadron beam to a target tissue.

FIG. 7 shows (a) to (d) magnetic data acquisition steps for imaging a volume by MRI, and (e) time sequences of emission of hadron pulses for visualizing the beam path according to an example embodiment of the present disclosure.

FIG. 8A shows an embodiment with synchronizations between emission of hadron beam pulses and MR-data acquisition steps MRe, MRp, and/or MRf, according to an example embodiment of the present disclosure.

FIG. 8B shows another embodiment with synchronizations between emission of hadron beam pulses and MR-data acquisition steps MRe, MRp, and/or MRf, according to another example embodiment of the present disclosure.

FIG. 9A shows an example of synchronized emission of a hadron pulse and of an MR-acquisition step, according to another example embodiment of the present disclosure.

FIG. 9B shows an example of a cut of a tissues traversed by a hadron beam from an upstream boundary to a target spot, with the localization of a sheath of ionized irradiated excitable atoms A1 indicated with dashed lines, according to an example embodiment of the present disclosure.

FIG. 9C shows a corresponding Eₗₒₛₛ curve of the hadron beam represented in the examples of FIGS. 9B, 9D, and 9E.

FIG. 9D shows a schematic representation of an MRI image obtained according to an example embodiment of the present disclosure with the beam path of the hadron beam visible as a hyposignal in case of a match between the positions of the Bragg peak and of the target spot.

FIG. 9E shows a schematic representation of another MRI image obtained according to another example embodiment of the present disclosure with the beam path of the hadron beam visible as a hyposignal in case of a mismatch between the positions of the Bragg peak and of the target spot.

FIG. 10A illustrates relaxation of an excited atom from an excited state at 90° with the spin of the atoms being in phase, according to an example embodiment of the present disclosure. M/M0 represents the relative magnetic moment, with M0 being the maximum value of said magnetic moment, M.

FIG. 10B illustrates the effect of a magnetic gradient along X2 on the phases of the spins of the example of FIG. 10A, according to an example embodiment of the present disclosure.

FIG. 10C illustrates the effect of a magnetic gradient along X3 on the frequencies of the spins of the example of FIG. 10A, according to an example embodiment of the present disclosure.

FIG. 11 shows a flowchart of a method according to an example embodiment of the present disclosure.

FIG. 12 shows magnetic data acquisition steps for imaging a volume by MRI, and time sequences of emission of hadron pulses for visualizing the tissues traversed by a hadron beam, for avoiding any artefacts caused by the irradiated excitable atoms A1, according to an example embodiment of the present disclosure.

The figures are not drawn to scale. Generally, identical components are denoted by the same reference numerals in the figures.

### DETAILED DESCRIPTION

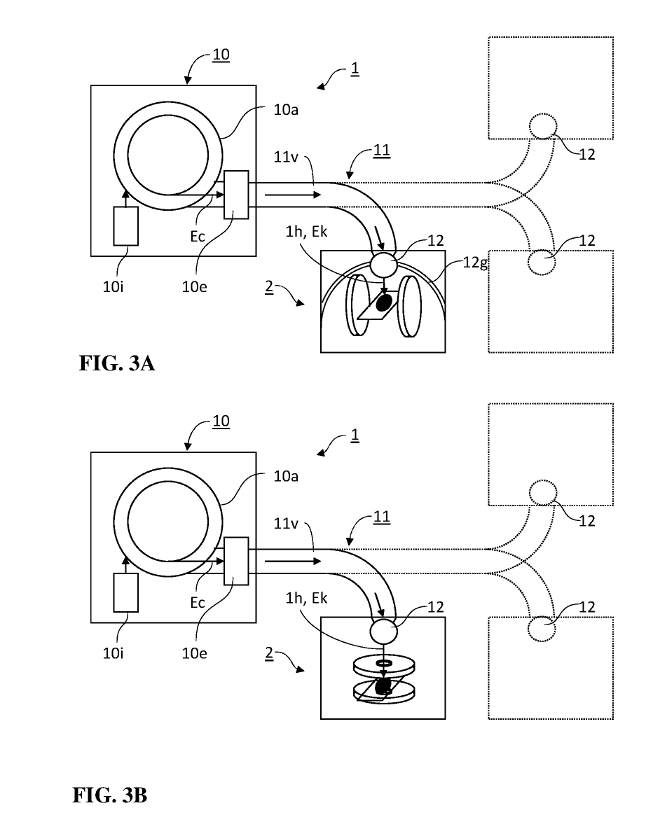

FIGS. 3A and 3B illustrate two examples of a medical apparatus comprising a hadron therapy device 1 coupled to a magnetic resonance imaging device (MRI) 2 according to embodiments of the present disclosure. A hadron therapy device, an MRI, and the combination of the two are described in greater details in the following description.

### Hadron Therapy Device

Hadron therapy is a form of external beam radiotherapy using beams 1h of energetic hadrons. FIGS. 3A, 3B, 4A, 6A, and 6B show a hadron beam 1h directed towards a target spot 40s in a target tissue 40 of a subject of interest. Target tissues 40 of a subject of interest typically include cancerous cells forming a tumour. During a hadron therapy session, a hadron beam of initial energy, Ek, with k=0 or 1, may irradiate one or more target spots within the target tissue, such as a tumour, and destroy the cancerous cells included in the irradiated target spots, reducing the size of the treated tumour by necrosis of the irradiated tissues.

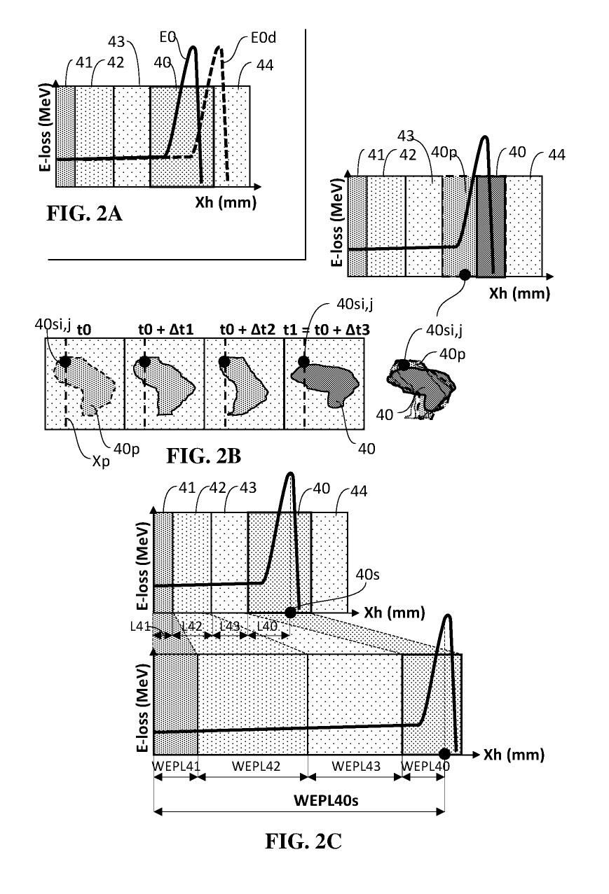

The subject of interest may comprise a plurality of materials including organic materials. For example, the subject of interest may comprise a plurality of tissues m, with m=40-44 as shown in the example of FIGS. 2A, 2B, and 2C, that may be, for example, skin, fat, muscle, bone, air, water (and/or blood), organ, tumour, or the like. For example, the target tissue 40 may be a tumour.

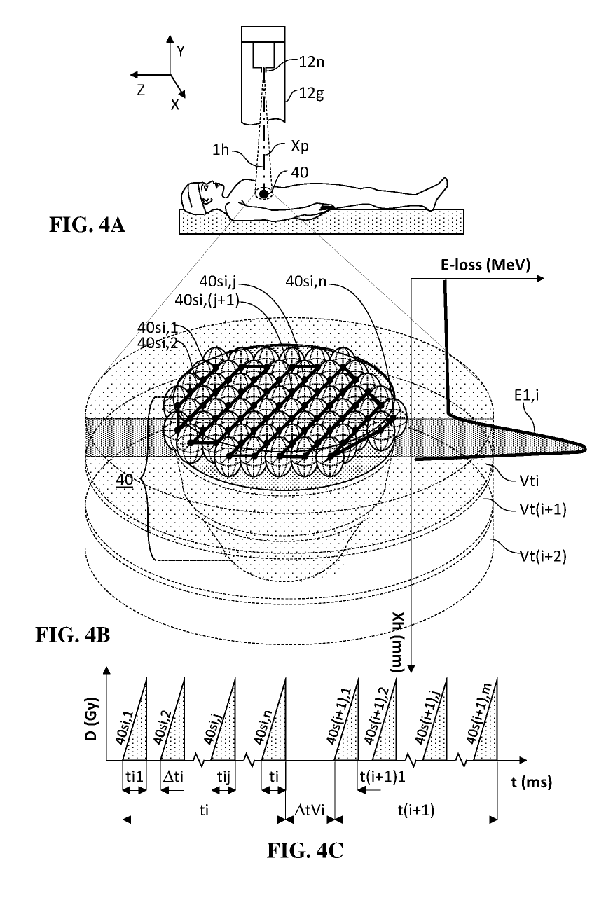

A hadron beam 1h traversing an organic body along a beam path, Xp, generally loses most of its energy at a specific distance of penetration along the beam path, Xp. As illustrated in FIGS. 2A, 2B, 2C, and 4B, said specific distance of penetration may correspond to the position of the Bragg peak, observed when plotting the energy loss per unit distance [MeVg⁻¹cm⁻²], Eₗₒₛₛ, of a hadron beam as a function of the distance, xh, measured along the beam path, Xp. Unlike other forms of radiation therapies, a hadron beam may therefore deliver a high dose of energy at a very specific location within a target tissue corresponding to the position of the Bragg peak. The position of the Bragg peak may depend mainly on the initial energy, Ek, of the hadron beam (i.e., before traversing any tissue) and on the nature and thicknesses of the traversed tissues. The hadron dose delivered to a target spot may depend on the intensity of the hadron beam and on the time of exposure. The hadron dose may be measured in Grays (Gy), and the dose delivered during a treatment session is usually of the order of one to several Grays (Gy).

A hadron is a composite particle made of quarks held together by strong nuclear forces. Typical examples of hadrons may include protons, neutrons, pions, heavy ions, such as carbon ions, and the like. In hadron therapy, electrically charged hadrons are often used. For example, the hadron may be a proton, and the corresponding hadron therapy may be referred to as proton therapy. Accordingly, in the following description, unless otherwise indicated, any reference to a proton beam and/or proton therapy may apply to a hadron beam and/or hadron therapy in general.

A hadron therapy device 1 generally comprises a hadron source 10, a beam transport line 11, and a beam delivery system 12. Charged hadrons may be generated from an injection system 10i, and may be accelerated in a particle accelerator 10a to build up energy. Suitable accelerators may include, for example, a cyclotron, a (synchro-)cyclotron, a synchrotron, a laser accelerator, or the like. For example, a (synchro-)cyclotron may accelerate charged hadron particles from a central area of the (synchro-)cyclotron along an outward spiral path until the particles reach the desired output energy, Ec, whence they may be extracted from the (synchro-)cyclotron. Said output energy, Ec, reached by a hadron beam when extracted from the (synchro-)cyclotron is typically comprised between 60 MeV and 400 MeV, e.g., between 210 MeV and 250 MeV. The output energy, Ec, may be, but is not necessarily, the initial energy, Ek, of the hadron beam used during a therapy session. For example, Ek may be equal to or lower than Ec, such that Ek≤Ec. An example of a suitable hadron therapy device may include, but is not limited to, a device described in U.S. Pat. No. 4,870,287,the entire disclosure of which is incorporated herein by reference as representative of a hadron beam therapy device used in the present disclosure.

The energy of a hadron beam extracted from a (synchro-)cyclotron may be decreased by energy selection means 10e, such as energy degraders or the like, positioned along the beam path, Xp, downstream of the (synchro-)cyclotron. Energy selection means 10e may decrease the output energy, Ec, down to any value of Ek, including down to nearly 0 MeV. As discussed supra, the position of the Bragg peak along a hadron beam path, Xp, traversing specific tissues may depend on the initial energy, Ek, of the hadron beam. By selecting the initial energy, Ek, of a hadron beam intersecting a target spot 40s located within a target tissue, the position of the Bragg peak may be controlled to correspond to the position of the target spot.

A hadron beam may also be used for characterizing properties of tissues. For example, images may be obtained with a hadron radiography system (HRS), for example, a proton radiography system (PRS). The doses of hadrons delivered to a target spot for characterization purposes, however, may be considerably lower than the doses delivered during a hadron therapy session, which, as discussed supra, may be of the order of 1 to 10 Gy. The doses of delivered hadrons of HRS for characterization purposes are typically of the order of 10⁻³ to 10⁻¹ Gy (i.e., one to four orders of magnitude lower than doses typically delivered for therapeutic treatments). These doses may have no significant therapeutic effects on a target spot. Alternatively or concurrently, a treatment hadron beam delivered to a small set of target spots in a target tissue may be used for characterization purposes. The total dose delivered for characterization purposes may be insufficient to treat a target tissue.

As illustrated in FIGS. 3A and 3B, downstream of the hadron source, a hadron beam of initial energy, Ek, may be directed to the beam delivery system 12 through a beam transport line 11. The beam transport line may comprise one or more vacuum ducts, 11v, and a plurality of magnets for controlling the direction of the hadron beam and/or for focusing the hadron beam. The beam transport line may also be adapted for distributing and/or selectively directing the hadron beam from a single hadron source 10 to a plurality of beam delivery systems for treating several patients in parallel.

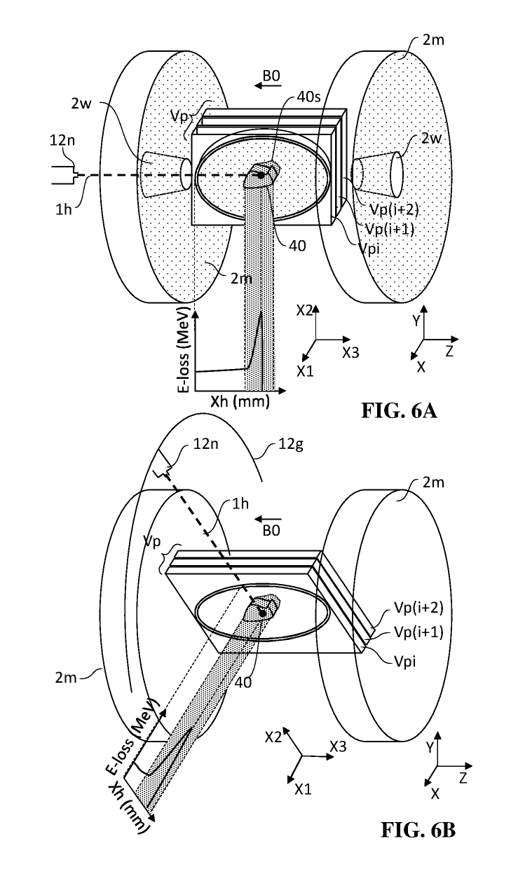

The beam delivery system 12 may further comprise a nozzle 12n for orienting a hadron beam 1h along a beam path, Xp. The nozzle may be fixed or mobile. Mobile nozzles are generally mounted on a gantry 12g, as illustrated schematically in the examples of FIGS. 4A and 6B. A gantry may be used for varying the orientation of the hadron outlet about a circle centred on an isocentre and normal to an axis, Z, which may be horizontal. In supine hadron treatment devices, the horizontal axis, Z, may be selected parallel to a patient lying on a couch (i.e., the head and feet of the patient are aligned along the horizontal axis, Z).

The nozzle 12n and the isocentre define a path axis, Xn, whose angular orientation depends on the angular position of the nozzle in the gantry. By means of magnets positioned adjacent to the nozzle, the beam path, Xp, of a hadron beam 1h may be deviated with respect to the path axis, Xn, within a cone centred on the path axis and having the nozzle as apex (as depicted, for example, in FIG. 4A). Advantageously, this may allow a volume of target tissue centred on the isocentre to be treated by a hadron beam without changing the position of the nozzle within the gantry. The same applies to fixed nozzles with the difference that the angular position of the path axis may be fixed.

A target tissue to be treated by a hadron beam in a device provided with a gantry must generally be positioned near the isocentre. Accordingly, the couch or any other support for the patient may be moved; for example, it may typically be translated over a horizontal plane (X, Z), wherein X is a horizontal axis normal to the horizontal axis, Z, and translated over a vertical axis, Y, normal to X and Z, and may also be rotated about any of the axes X, Y, Z, so that a central area of the target tissue may be positioned at the isocentre.

To assist in the correct positioning of a patient with respect to the nozzle 12n according to a treatment plan previously established, the beam delivery system may comprise imaging means. For example, a conventional X-ray radiography system may be used to image an imaging volume, Vp, comprising the target tissue 40. The obtained images may be compared with corresponding images collected previously during the establishment of the treatment plan.

Depending on the pre-established treatment plan, a hadron treatment may comprise delivery of a hadron beam to a target tissue in various forms, including, for example, pencil beam, single scattering, double scattering, uniform scattering, and the like. Embodiments of the present disclosure may apply to all hadron therapy techniques. FIG. 4B illustrates schematically a pencil beam technique of delivery. As depicted in FIG. 4B, hadron beam of initial energy, Ek,1, may be directed to a first target spot 40s1,1, during a pre-established delivery time. The hadron beam may then be moved to a second target spot 40s1,2, during a pre-established delivery time. The process may be repeated on a sequence of target spots 40s1,j to scan a first iso-energy treatment volume, Vt1, following a pre-established scanning path. A second iso-energy treatment volume, Vt2, may be scanned spot-by-spot following a similar scanning path with a hadron beam of initial energy, Ek,2. As many iso-energy treatment volumes, Vti, as necessary to treat a given target tissue 40 may thus be irradiated following a similar scanning path. A scanning path may include several passages over a same scanning spot 40si,j. The iso-energy treatment volumes, Vti, may be volumes of target tissues which may be treated with a hadron beam of initial energy, Ek,i. The iso-energy treatment volumes, Vti, may be slice shaped, with a thickness corresponding approximately to the breadths of the Bragg peaks at the values of the initial energy, Ek,i, of the corresponding hadron beams, and with main surfaces of area only limited by the opening angle of the cone centred on the path axis, Xn, enclosing the beam paths, Xp, available for a given position of the nozzle in the gantry or in a fixed nozzle device. In embodiments with a homogeneous target tissue, the main surfaces may be substantially planar as illustrated in FIG. 4B. In embodiments where both target tissue 40 and upstream tissues 41-43 are not homogeneous in nature and thickness, the main surfaces of an iso-energy volume, Vti, may be bumpy. The egg-shaped volumes in FIG. 4B schematically illustrate the volumes of target tissue receiving a therapeutic dose of hadron by exposure of one target spot 40si,j to a beam of initial energy Ek,i.

The dose, D, delivered to a target tissue 40 is illustrated in FIG. 4C. As discussed supra, the dose delivered during a treatment session is usually of the order of one to several Grays (Gy). It may depend on the doses delivered to each target spot 40si,j, of each iso-energy treatment volume, Vti. The dose delivered to each target spot 40sij may depend on the intensity, I, of the hadron beam and on the irradiation time tij on said target spot. The dose, Dij, delivered to a target spot 40si,j may therefore be the integral, Dij=∫I dt, over the irradiation time tij. A typical dose, Dij, delivered to a target spot 40si,j is generally of the order of 0.1-20 cGy. The dose, Di, delivered to an iso-energy treatment volume, Vti, may be the sum over the n target spots scanned in said iso-energy treatment volume of the doses, Dij, delivered to each target spot, Di=ΣDij, for j=1 to n. The total dose, D, delivered to a target tissue 40 may thus be the sum over the p irradiated iso-energy treatment volumes, Vti, of the doses, Di, delivered to each energy treatment volume, D=ΣDi, for i =1 to p. The dose, D, of hadrons delivered to a target tissue may therefore be controlled over a broad range of values by controlling one or more of the intensity, I, of the hadron beam, the total irradiation time tij of each target spot 40si,j, and/or the number of irradiated target spots 40si,j. Once a patient is positioned such that the target tissue 40 to be treated is located at the approximate position of the isocentre, the duration of a hadron treatment session may depend on the values of:
the irradiation time, tij, of each target spot 40si,j,
the scanning time, Δti, for directing the hadron beam from a target spot 40si,j to an adjacent target spot 40si(j +1) of a same iso-energy treatment volume, Vti,
the number n of target spots 40si,j scanned in each iso-energy treatment volume, Vti,
the time, ΔtVi, required for passing from a last target spot 40si,n scanned in an iso-energy treatment volume, Vti, to a first target spot 40s(i+1),1 of the next iso-energy treatment volume, Vt(i+1), and/or
the number of iso-energy treatment volumes, Vti, in which a target tissue 40 may be enclosed.

The irradiation time, tij, of a target spot 40si,j is generally of the order of 1-20 ms. The scanning time, Δti, between successive target spots in a same iso-energy treatment volume may be very short, of the order of 1 ms. The time, ΔtVi, required for passing from one iso-energy treatment volume, Vti, to a subsequent iso-energy treatment volume, Vt(i+1), may be slightly longer because, for example, it may require changing the initial energy, Ek, of the hadron beam. The time required for passing from one volume to a subsequent volume is generally of the order of 1-2 s.

As evidenced in FIGS. 2A and 2B, an accurate determination of the initial energy, Ek, of a hadron beam may be important because, if the position of the Bragg peak does not correspond to the actual position of the target tissue 40, substantial doses of hadrons may be delivered to healthy, sometimes vital, organs and may possibly endanger the health of a patient. The position of the Bragg peak may depend on the initial energy, Ek, of the hadron beam and/or on the nature and thicknesses of the traversed tissues. Besides determining the position of the target tissue within a patient, the computation of the initial energy, Ek, of a hadron beam yielding a position of the Bragg peak corresponding to the precise position of the target tissue may also require the preliminary characterization of the tissues traversed until reaching the target tissue 40. This characterization may be performed during a treatment plan established before (e.g., generally several days before) the actual hadron treatment. The actual hadron treatment may be divided in several sessions distributed over several weeks. A typical treatment plan may start by the acquisition of data, e.g., generally in the form of images of the subject of interest with a CT scan. The images thus acquired by a CT scan may be characterized, for example, by performing one or more of the following steps:
identifying the nature of the tissues represented on the images as a function of the X-rays absorption power of the tissues, e.g., based on the comparison of shades of grey of each tissue with a known grey scale; for example, a tissue may be one of fat, bone, muscle, water, air, or the like;
measuring the positions and thicknesses of each tissue along one or more hadron beam paths, Xp, from the skin to the target tissue;
based on their respective nature, attributing to each identified tissue a corresponding hadron stopping power ratio (HSPR);
calculating a tissue water equivalent path length, WEPLm, of each tissue m, with m=40 to 44 in the illustrated examples of FIGS. 2A and 2B, upstream of and including the target tissue, based on their respective HSPR and thicknesses;
adding the determined WEPLm of all tissues m to yield a WEPL40s of a target spot 40s located in the target tissue 40, said WEPL40s corresponding to the distance travelled by hadron beam from the skin to the target spot 40s; and
based on the WEPL40s, calculating the initial energy Ek of a hadron beam required for positioning the Bragg peak of the hadron beam at the target spot 40s.
Said process steps may be repeated for several target spots defining the target tissue.

### Magnetic Resonance Imaging Device

A magnetic resonance imaging device 2 (MRI) generally implements a medical imaging technique based on the interactions of excitable atoms present in an organic tissue of a subject of interest with electromagnetic fields. When placed in a strong main magnetic field, B0, the spins of the nuclei of said excitable atoms typically precess around an axis aligned with the main magnetic field, B0, resulting in a net polarization at rest that is parallel to the main magnetic field, B0. The application of a pulse of radio frequency (RF) exciting magnetic field, B1, at the frequency of resonance, fL, called the Larmor frequency, of the excitable atoms in said main magnetic field, B0, may excite said atoms by tipping the net polarization vector sideways (e.g., with a so-called 90° pulse, B1-90) or to angles greater than 90° and even reverse it at 180° (e.g., with a so-called 180° pulse, B1-180). When the RF electromagnetic pulse is turned off, the spins of the nuclei of the excitable atoms generally return progressively to an equilibrium state yielding the net polarization at rest. During relaxation, the transverse vector component of the spins typically produces an oscillating magnetic field inducing a signal, which may be collected by antennas 2a located in close proximity to the anatomy under examination.

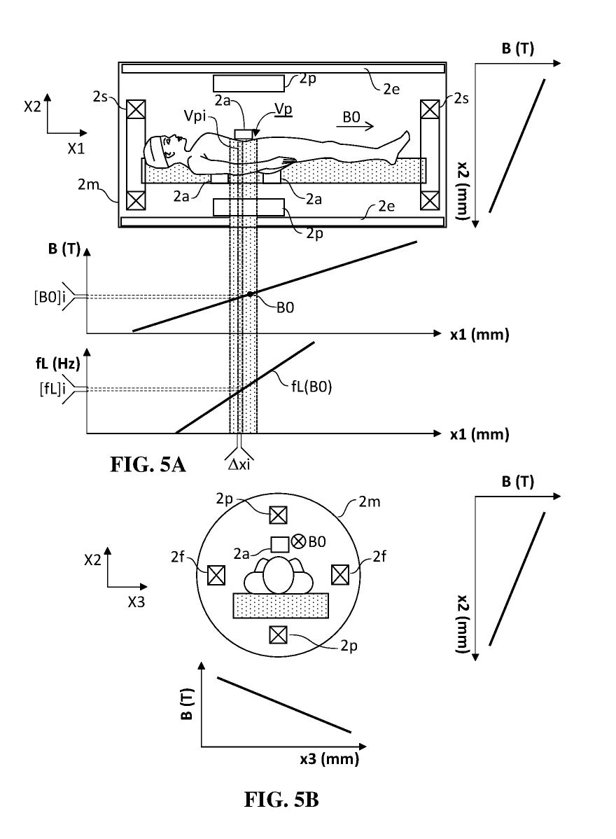

As shown in FIGS. 5A, 5B, 6A, and 6B, an MRI 2 usually comprises a main magnet unit 2m for creating a uniform main magnetic field, B0; radiofrequency (RF) excitation coils 2e for creating the RF-exciting magnetic field, B1; X1-, X2-, and X3-gradient coils, 2s, 2p, 2f, for creating magnetic gradients along the first, second, and third directions X1, X2, and X3, respectively; and antennas 2a, for receiving RF-signals emitted by excited atoms as they relax from their excited state back to their rest state. The main magnet may produce the main magnetic field, B0, and may be a permanent magnet or an electro-magnet (e.g., a supra-conductive magnet or not). An example of a suitable MRI includes, but is not limited to, a device described in U.S. Pat. No. 4,694,836, the entire disclosure of which is incorporated herein by reference as representative of an MRI used in the present disclosure.

As illustrated in FIG. 5A, an imaging slice or layer, Vpi, of thickness, Δxi, normal to the first direction, X1, can be selected by creating a magnetic field gradient along the first direction, X1. In FIG. 5A, the first direction, X1, is parallel to the axis Z defined by the lying position of the patient, yielding slices normal to said axis Z. In some embodiments, the first direction, X1, may be any direction, e.g., transverse to the axis Z, with slices extending at an angle with respect to the patient. As further shown in FIG. 5A, because the Larmor frequency, fL, of an excitable atom generally depends on the magnitude of the magnetic field it is exposed to, sending pulses of RF exciting magnetic field, B1, at a frequency range, [fL]i, may excite exclusively the excitable atoms which are exposed to a magnetic field range, [B0]i, which may be located in a slice or layer, Vpi, of thickness, Δxi. By varying the frequency bandwidth, [fL]i, of the pulses of RF exciting magnetic field, B1, the width, Δxi, and position of an imaging layer, Vpi, may be controlled. By repeating this operation on successive imaging layers, Vpi, an imaging volume, Vp, may be characterized and imaged.

To localize the spatial origin of the signals received by the antennas on a plane normal to the first direction, X1, magnetic gradients may be created successively along second and third directions, X2, X3, wherein X1⊥X2⊥X3, by activating the X2-, and X3-gradient coils 2p, 2f, as illustrated in FIG. 5B. Said gradients may provoke a phase gradient, Δφ, and a frequency gradient, Δf, in the spins of the excited nuclei as they relax, which may allow spatial encoding of the received signals in the second and third directions, X2, X3. A two-dimensional matrix may thus be acquired, producing k-space data, and an MR image may be created by performing a two-dimensional inverse Fourier transform. Other modes of acquiring and creating an MR image may be utilized concurrently with or alternatively to the mode described above.

The main magnetic field, B0, may be between 0.2 T and 7 T, e.g., between 1 T and 4 T. The radiofrequency (RF) excitation coils 2e may generate a magnetic field at a frequency range, [fL]i, around the Larmor frequencies, fL, of the atoms comprised within a slice of thickness, Δxi, and exposed to a main magnetic field range [B0i]. For atoms of hydrogen, the Larmor frequency per magnetic strength unit is approximately fL/B=42.6 MHz T⁻¹. For example, for hydrogen atoms exposed to a main magnetic field, B0=2 T, the Larmor frequency is approximately fL=85.2 MHz.

The MRI may be any of a closed-bore, open-bore, or wide-bore MRI type. A typical closed-bore MRI has a magnetic strength of 1.0 T through 3.0 T with a bore diameter of the order of 60 cm. An open-bore MRI, as illustrated in FIGS. 6A and 6B, has typically two main magnet poles 2m separated by a gap for accommodating a patient in a lying position, sitting position, or any other position suitable for imaging an imaging volume, Vp. The magnetic field of an open-bore MRI is usually between 0.2 T and 1.0 T. A wide-bore MRI is a kind of closed-bore MRI having a larger diameter.

### Hadron Therapy Device+MRI

As discussed previously with reference to FIG. 2B, the position and morphology of a target tissue 40 may evolve between a time, t0, of establishment of a treatment plan and a time, t1=t0+Δt3, of a treatment session, which may be separated by several days or weeks. A target spot 40si,j identified in the treatment plan as belonging to the target tissue 40p may not belong to the target tissue 40 anymore at the time, t0+Δt3, of the treatment session. The irradiation of said target spot may harm healthy tissues 43 instead of target tissues 40.

To avoid such incidents, a hadron therapy device (PT) 1 may be coupled to an imaging device, such as a magnetic resonance imaging device (MRI) 2. Such coupling may raise a number of challenges to overcome. For example, the correction of a hadron beam path, Xp, within a strong magnetic field, B0, of the MRI is a well-researched problem with proposed solutions.

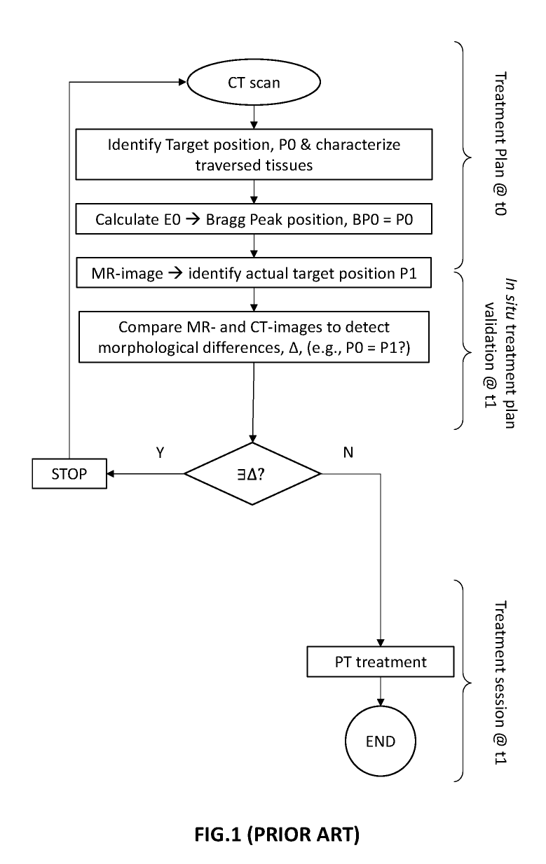

A PT-MRI apparatus may allow the morphologies and positions of the target tissue and surrounding tissues to be visualized, for example, on the day, t0+Δt3, of the treatment session for comparison with the corresponding morphologies and positions acquired during the establishment of a treatment plan at time, t0. As illustrated in the flowchart of FIG. 1, in cases having a discrepancy of the tissues morphologies and positions between the establishment of the treatment plan at time, t0, and the treatment session at time, t0+Δt3, the treatment session may be interrupted and a new treatment plan may be established with the definition of new target spots corresponding to the actual target tissue 40 to be irradiated by hadron beams of corrected energies and directions (in the example of FIG. 1, this procedure is represented by diamond box “∃Δ?” →Y→“STOP”). This represents a major improvement over carrying out a hadron therapy session based solely on information collected during the establishment of the treatment plan at time, t0, which may be obsolete at the time, t0+Δt3, of the treatment session.

Embodiments of the present disclosure may further improve the efficacy of a PT-MRI apparatus by providing the information required for correcting in situ the initial energies, Ek, and beam path, Xp, directions of the hadron beams, in case a change of morphology or position of the target tissue were detected. This may allow the treatment session to take place in spite of any changes detected in the target tissue 40.

The MRI used in embodiments of the present disclosure may be any of a closed-bore, open-bore, or wide-bore MRI type described above. An open MRI may provide open space in the gap separating the two main magnet poles 2m for orienting a hadron beam in almost any direction. Alternatively, openings or windows 2w transparent to hadrons may be provided on the main magnet units, as illustrated in the example of FIG. 6A. This configuration may allow the hadron beam to be parallel to B0. In another embodiment, a hadron beam may be oriented through the cavity of the tunnel formed by a closed bore MRI, or an annular window transparent to hadrons may extend parallel to a gantry substantially normal to the axis Z, over a wall of said tunnel, such that hadron beams may reach a target tissue with different angles. In embodiments where a fixed nozzle is used, the size of such opening or window may be reduced accordingly.

### MRI Imaging of Tissues and of Hadron Beam

As described above, the principle of acquisition of an MR image is based on the interactions of excitable atoms A0 present in a target tissue in response to an exciting RF-magnetic field B1-sequence. A hadron beam may interact with the excitable atoms, yielding irradiated excitable atoms A1. Absorption of a ionizing radiation such as a hadron beam by living cells generally directly disrupts atomic structures, producing chemical and biological changes and indirectly disrupts through radiolysis of cellular water and generation of reactive chemical species, by stimulation of oxidases and nitric oxide synthases. As of the date of filing, the hadrons of a hadron beam typically cannot be visualized directly by MR imaging techniques. However, the effects on the irradiated excitable atoms A1 affected by the passage of the hadron beam may modify the RF signals emitted by the excited atoms as they relax. The irradiated excitable atoms A1 may therefore create artefacts that disrupt the MR image because the irradiated excitable atoms emit RF-signals during relaxation, which are different from the signals they would have emitted had they not been irradiated. If uncontrolled, these artefacts may be dangerous as they may yield MR images unrepresentative of reality. Embodiments of the present disclosure may use such artefacts to enable visualization of the path or trail created by the hadron beam, which may be representative of the beam path. Accordingly, the position of the hadron beam path may be identified indirectly.

Irradiation by a hadron beam may have one or more of the following effects on the tissues it traverses. First, irradiated excitable atoms A1 may be ionized by the passage of a hadron beam. The ionization lifetime of the irradiated excitable atoms A1, however, is generally short, ceasing within micro-seconds after the end of the irradiation. Second, the magnetic susceptibility of excitable atoms A0 may be modified by the passage of a hadron beam, yielding irradiated excitable atoms A1. Because of their differing magnetic susceptibility, the irradiated excitable atoms A1 typically respond differently to the exciting magnetic field B1-sequence. For example, the Larmor rest frequency, fLm0 of an excitable atom A0, such as hydrogen, in a tissue m exposed to a main magnetic field, B0, may shift to a value, fLm1, of a Larmor irradiated frequency of an irradiated excitable atom A1, when said excitable atom was irradiated by a hadron beam. The shift, ΔfLm=|fLm1−fLm0|, may explain, at least in part, why RF-signals emitted during relaxation by irradiated excitable atoms A1 differ from the ones emitted by non-irradiated excitable atoms, A0.

The concentration of irradiated excitable atoms A1 may be a function of the energy deposited by said hadron beam in the tissues it traverses. As illustrated in the examples of FIGS. 2A, 2B, and 2C, a hadron beam generally deposits almost all its energy at the level of the Bragg peak, which may be quite narrow. The ionization of irradiated excitable atoms, A1, and the magnetic susceptibility of the excitable atoms A1 on and adjacent to the hadron beam path may therefore vary most at the level of the Bragg peak, resulting from a higher concentration of irradiated excitable atoms A1 at said level of the Bragg peak. In addition to visualizing indirectly the beam path of a hadron beam, it may therefore be possible to localize the position of the Bragg peak, where the presence of irradiated excitable atoms A1 is marked most.

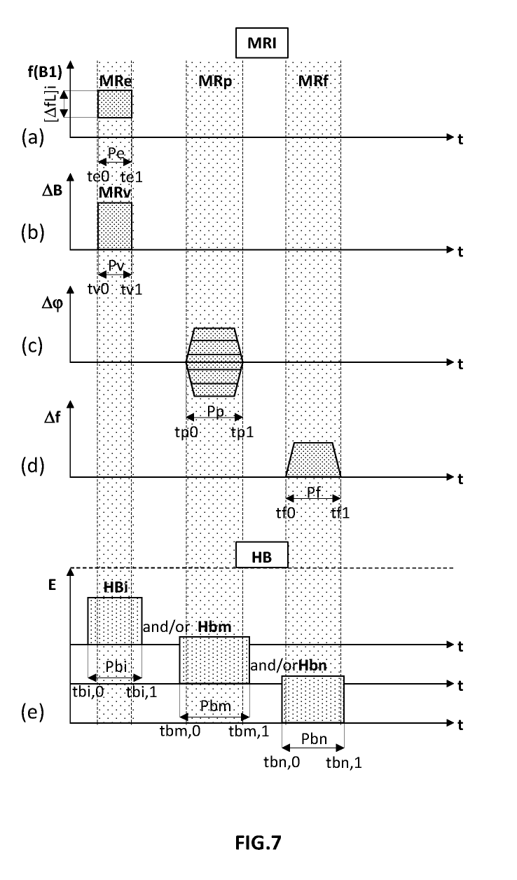

Acquisition of magnetic resonance data by an MRI for imaging a volume, Vp, according to one embodiment the present disclosure may comprise the following MR-data acquisition steps illustrated in FIG. 7, steps (a)-(d).
(A) an excitation step (MRe) illustrated in FIG. 7, step (a), for exciting the spin of the nuclei of excitable atoms A0, by creating an oscillating electromagnetic field, B1, at a given RF frequency range [fL]i corresponding to the Larmor frequencies of the excitable atoms located within the imaging layer, Vpi, during an excitation period, Pe=(te1−te0), wherein te0 and te1 are the times of the beginning and end of the excitation step, respectively;
(B) a layer selection step (MRv) illustrated in FIG. 7, step (b), for selecting an imaging layer, Vpi, of the imaging volume, Vp, of thickness, Δxi, measured along the first direction, X1 including creating a magnetic field gradient in a first direction, X1;
(C) a phase gradient step (MRp) illustrated in FIG. 7, step (c), for localising along the second direction, X2, the origin of RF signals received by the antennas during relaxation of the excited spins, including creating magnetic field gradients in a second direction, X2, normal to the first direction, X1, (X1⊥X2), during a period Pp=(tp1−tp0), wherein tp0 and tp1 are the times of the beginning and end of the phase gradient step, respectively, with tf0>tp1; and
(D) a frequency gradient step (MRf) illustrated in FIG. 7, step (d), for localising along the third direction, X3, the origin of RF signals received by the antennas during relaxation of the excited spins including creating magnetic field gradients in a third direction, X3, normal to the first and second directions, (X1⊥X2⊥X3), during a period Pf=(tf1-31 tf0), wherein tf0 and tf1 are the times of the beginning and end of the frequency gradient step, respectively, with tf0 >tp1.

The imaging volume, Vp, may generally be divided into several imaging layers, Vpi, sizes of which may be restricted along three dimensions by creating a magnetic gradient along, one, two, or three of the first, second, and third directions, X1, X2, X3. The thickness of the imaging volume may thus be controlled along said first, second, or third directions, X1, X2, X3, to define a slice (i.e., restricted over one direction only), an elongated prism (i.e., restricted over two directions), or a box (i.e., restricted over the three directions X1, X2, X3.

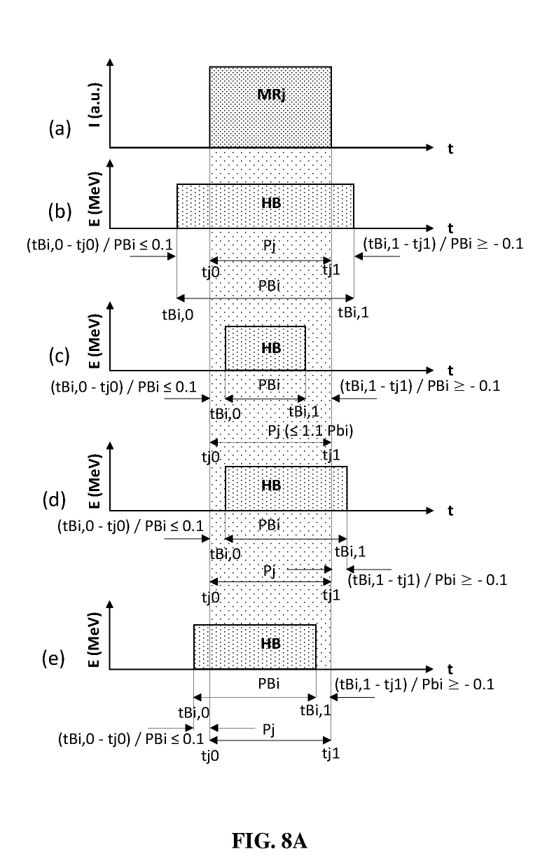

By synchronizing these steps with the emission of N pulses of a hadron beam, the visualization of the beam path of the hadron beam may be possible by detecting the effects the hadron beam has on the irradiated excitable atoms A1. In particular, a hadron beam having an initial energy, E0, may be directed along a beam path intersecting said target body in the imaging layer, Vpi, in a number, N, of hadron pulses of pulse periods, Pbi, wherein, N is an integer greater than 0. As illustrated in FIGS. 7, step (e), and in FIGS. 8A and 8B, the MR data acquisition steps may be synchronized with the N hadron pulses, such that at an MR-period, Pj, (with j=e, f, and/or p, of one or more of the excitation step, Pe, the phase gradient step, Pp, and the frequency gradient step, Pt) overlaps with and does not exceed the pulse period, Pbi, by more than 10%, Pj≤1.1 Pbi. Furthermore, the MR-period, Pj, may be out of phase with respect to the pulse period Pbi of an overlapping hadron pulse by no more than 10%, such that (tBi,0−tj0)/PBi≤0.1, and (tBi,1−tj1)/PBi≥−0.1, with j=e, f, and/or p.

The MR-data acquisition steps may comprise additional sequences, such as:
a layer selection step simultaneously with an excitation step,
a phase gradient step, and/or
a frequency gradient step.
   These additional steps may be set in sequences identical to, or different from, the sequence illustrated in FIG. 7, and their periods, Pj, can vary. The acquisition of magnetic resonance data and the emission of hadron pulses may also be synchronized with said additional steps as defined above in order to strengthen the signal.

The synchronizations with the excitation step (MRe), phase gradient step (MRp) and/or frequency gradient step (MRI) are discussed in greater detail in the following description.

### Excitation Step (MRe)

The excitation step may comprise creating pulses of an excitation electromagnetic field, B1, with the RF unit 2e during an excitation period, Pe, oscillating at a RF-frequency range, [fL]i. The excitation step MRe is represented in FIGS. 7, step (a), and in FIGS. 8A and 8B as square signals with clearly defined upper and lower frequency boundaries defining the bandwidth of the RF-frequency range, [fL]i. In some embodiments, the RF signal is not perfectly square. The upper and lower boundaries of an RF-frequency range, [fL]i., may be the frequencies corresponding to 20% of the maximum intensity of the signal. For a square signal, then, the upper and lower boundaries may be the same as depicted in the example of FIG. 7, step (a). The boundaries for a bell shaped signal may be similarly defined.

The ionization of the hydrated electrons of the irradiated excitable atoms Al may form a sheath around the hadron beam, shielding the excitable atoms included in said sheath from the excitation from the RF-electromagnetic field, B1, during an excitation step. After the excitation step, RF signals emitted by the excited atoms A0 located outside the sheath upon relaxing may be collected and localized during the phase gradient and frequency gradient steps as explained above. The irradiated excitable atoms A1 located within the sheath, however, generally do not emit relaxation related signals that are as strong as those of the atoms A0, since they were excited less, if at all, during the excitation step. Consequently, the volume comprised within the sheath may appear in an MR image as a hyposignal.

Since the ionization lifetime of the hydrated electrons is generally very short, e.g., of the order of the us, the excitation step and hadron beam emission may be synchronized such that the excitation step coincides with the emission of hadron beam during at least 90% of the excitation period, Pe. As shown in FIG. 8A, steps (b) to (e), and in FIG. 8B, step (f), the hadron beam may be longer than the excitation step. The period PBi of a hadron beam pulse, however, may be minimized, since said hadron beam pulse is generally not part of the treatment plan and usually has no therapeutic purpose. For example, said hadron beam pulse may be solely for localizing the actual position of the beam path and of the Bragg peak of the hadron beam. Accordingly, the hadron pulse may be as short as possible in order to destroy less, if any, healthy tissue in case the initial energy, E0, must be corrected, and/or in order to not disrupt the treatment plan in case the initial energy, E0, is correct. A hadron beam pulse overlapping completely and extending beyond an excitation period, Pe, as illustrated in FIG. 8, step (b), may therefore seldom be used. Therefore, the hadron beam pulse may generally be shorter than the excitation period, Pe, as illustrated in FIG. 8, step (c). Moreover, the hadron pulse period, PBi, may overlap with the excitation step during at least 90% of the excitation period, Pe. FIG. 8, steps (d) and (e) depict examples where the hadron beam overlaps in a time scale with the excitation step on one side only. In some embodiments, the excitation period, Pe, may end close to the end of the pulse period, PBi, such that (tBi,1−te1)/PBi≥0, and such that (tBi,1−te1)/PBi≤0.3, e.g., (tBi,1−te1)/PBi ≤0.2, s(tBi,1−te1)/PBi ≤0, or (tBi,1−te1)/PBi=0. In some embodiments, the boundaries of the hadron beam pulse period, PBi, which is within the excitation step may be located no further from the corresponding boundary of the excitation step than by 10% of the hadron beam period, PBi.

FIG. 8B, step (f), shows an example embodiment comprising short hadron pulses wherein each pulse is substantially shorter than the MR-period Pj. The total burst period, PBt may be the sum, PBt=ΣPBi, of the periods PBi of each burst comprised within the MR-period Pj.

A hadron pulse usually does not consist of hadrons flowing continuously during the whole period PBi of the hadron pulse. Rather, a hadron pulse is generally formed by consecutive trains of hadrons. In some embodiments of the present disclosure, consecutive trains of hadrons separated from one another by a period of not more than 1.5 ms (milliseconds) may form a single hadron pulse. Inversely, if two trains of hadrons are separated by a period of more than 1.5 ms, they may belong to two separate hadron pulses. For example, a synchro-cyclotron emitting a 10 μs-hadron train every 1 ms during 10 ms may form a single hadron pulse of period PBi=10 ms. Typically, a hadron pulse may have a period, PBi, between 10 μs and 30 ms, depending on the type of hadron source used. In one example, the hadron beam pulse period, PBi, may be between 1 ms and 10 ms. In another embodiment, the hadron beam pulse period, PBi, may be between 5 and 20 ms. As discussed above with respect to the example of FIG. 4C, two consecutive hadron pulses may be separated from one another by a period, ΔPBi, for example, between 1 and 20 ms, e.g., between 2 and 10 ms.

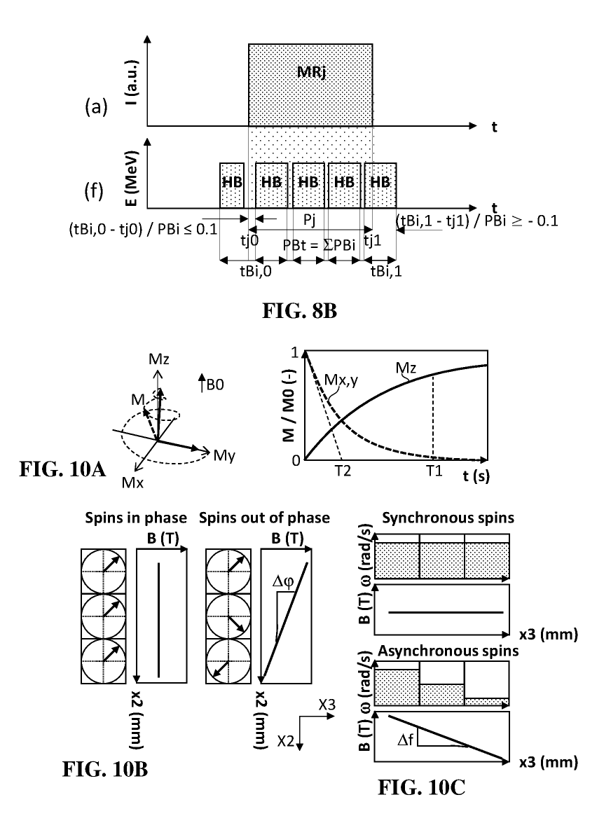

As illustrated in FIG. 9B, the irradiated excitable atoms, A1, ionized by the hadron beam may form a sheath 1s around the hadron beam 1h, with a higher level of ionization at the level of the Bragg peak. By synchronizing the emission of the hadron beam with the excitation step as described above (as, e.g., depicted in the example of FIG. 9A), the trail of the hadron beam may be visualized on an MR image as a hyposignal 1p. The target tissue 40 may be a tumour composed of cancerous cells and may comprise a target spot 40s (represented by a black spot in the examples of FIGS. 9B, 9C, 9D, and 9E) to be irradiated with a given dose according to a treatment plan. The hadron beam 1h may cross a number of healthy tissues 4-43 before reaching the target tissue 40 and the target spot 40s. The tissue 41 may, for example, be the skin of a patient. The thin dotted line in FIGS. 9B represents an irradiated volume surrounding the hadron beam 1h and defining a sheath 1s containing ionized atoms A1. Outside said irradiated volume, the excitable atoms A0 are generally little not affected significantly by the hadron beam. Tissue 44 may be a healthy tissue, possibly a vital tissue, located downstream of the target tissue 40, and may not be reached by the hadron beam.

FIG. 9C shows the energy loss curve of the hadron beam 1h of FIGS. 9B as it travels across the tissues until reaching the target spot in the target tissue. The hadron beam has an initial energy, EO, (i.e., before reaching the first tissue 41 along its beam path), which may have been determined previously during the establishment of a treatment plan. If the treatment plan was performed accurately, and if the relative positions and morphologies of the tissues 40-43 traversed by the hadron beam had not had changed since the establishment of the treatment plan, the Bragg peak of a hadron beam of initial energy, E0, generally falls at the position, P0, of the target spot 40s, as established during the treatment plan. This situation is illustrated in the example of FIG. 9D.

As discussed above, however, it is possible that the sizes and positions of the tissues traversed by a hadron beam changed between the day, t0, a treatment plan had been established and the day, t1, of a hadron therapy session. The example of FIG. 9E illustrates a case where the tissues 42 and 43 located upstream from the target tissue 40 have shrunk between times t0 and t1. Tissues 42 and 43 could, for example, be fat and muscles that can easily shrink during an illness. Consequently, the target tissue has moved closer to the upstream boundary of the treated anatomy, and the distance the hadron beam must travel across tissues until the actual position, P1, of the target spot has decreased accordingly, as depicted in the example of FIG. 9E. Irradiation of the tissues with a hadron beam of initial energy, E0, therefore may reach beyond the actual position of the target spot. As illustrated in FIG. 1, the identification of such mismatch between the planned position P0 and the actual position P1 in existing methods generally leads to the interruption of the treatment session and the establishment of a new treatment plan, which may waste precious time and resources.

### Phase Gradient Step (MRp)

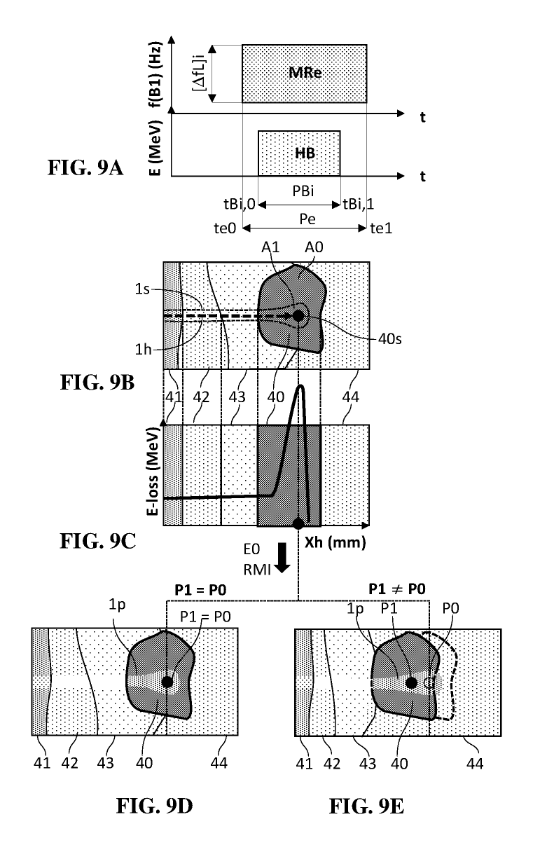

Absent an excitation electromagnetic field, B1, the net polarization vector of the excitable atoms A0 (generally hydrogen) of a tissue exposed to a main magnetic field, B0, parallel to the axis Z, is usually parallel to both B0 and Z, with a net polarization component, Mx,y, in the directions X and Y, which is generally zero as the spins precessing about the axis Z are out of phase and tend to compensate each other. As illustrated in FIG. 10A, upon excitation at their Larmor frequency with an excitation electromagnetic field, B1, the precessing angle of the spins may increase, yielding a decrease in the Z-component, Mz, of the net polarization vector (an excitation angle of 90° is illustrated in the example of FIG. 10A). With a proper RF-excitation sequence, the spins of the excited atoms may be brought into phase (as illustrated in the example of FIG. 10B and labeled “Spins in phase”). With the spins of the excited atoms precessing in phase, the net polarization component, Mx,y, may be non-zero. FIG. 10A illustrates an example of an excitable atom excited at 90° , yielding a zero Mz-component and maximum Mx,y-components, and then relaxing back to its rest state after the end of the excitation. The relaxation process and corresponding relaxation times, T1, T2 of this example are illustrated in the graph of FIG. 10A.

By applying a phase gradient along the second direction, X2, the spins of the excited atoms may be brought out of phase in a controlled manner, as shown in FIG. 10B and labeled “Spins out of phase.” Because the spins still precess at a same rotation rate, ω, the spins may remain out of phase even after the phase gradient step is terminated. At this stage, the spins of the atoms located in consecutive voxels aligned along the second direction, X2, may behave like a series of clocks indicating the times in different towns situated at different time zones of the world.

Because the irradiated excitable atoms A1 have a magnetic susceptibility different from the non-irradiated excited atoms A0, the former generally react differently to the phase gradient, and thus emit RF-signals upon relaxation which differ from the RF-signals sent by the non-irradiated excited atoms A0. By comparing an MR scan of the target tissue with and without a hadron beam (i.e., with and without irradiated excitable atoms A1), one may determine the trace left by the hadron beam. A comparison is often required because, in many cases, the hadron beam path may not be directly visible on an MR image. Instead, the MR image may look acceptable at first sight and therefore induce an operator into error who would not have identified the presence of artefacts created by the irradiated excitable atoms A0. In such conditions, a comparison of MR images taken with and without a hadron beam may reveal the location of the artefacts, and thus define the hadron beam path.

### Frequency Gradient Step (MRf)

Following the phase gradient step (MRp), the spins of excited atoms located in voxels aligned along the second direction, X2, are typically out of phase (as depicted in the example of FIG. 10B and labeled “Spins out of phase”) and precess at a same frequency (as depicted in the example of FIG. 10C and labeled “Synchronous spins”), such that all spins precess at a same rotation rate, ω. By applying a magnetic gradient along the third direction,

X3, the spins frequencies may be varied in a controlled manner along the third direction as shown in the example of FIG. 10C and labeled “Asynchronous spins.”

The MR data may then be acquired and an image formed based on the localization of the voxels whence phase and frequency specific RF-signals are emitted by excited atoms upon relaxation. Because the irradiated excitable atoms A1 have a magnetic susceptibility different from the non-irradiated excited atoms A0, the former generally react differently to the frequency gradient, and emit different RF-signals upon relaxation from the RF-signals sent by the non-irradiated excited atoms A0. The RF-signals emitted by irradiated excited atoms located in voxels traversed by the hadron beam therefore may differ from the RF-signals emitted by excited atoms in the same voxels absent a hadron beam. By comparing an MR scan of the target tissue with and without a hadron beam (i.e., with and without irradiated excitable atoms A1), one may determine the trace left by the hadron beam. The trail left by the hadron beam at the level of the Bragg peak is generally more intense, allowing localization the actual position of the Bragg peak of a hadron beam.

In order to capture the whole of a hadron beam path, the first direction, X1, defining the thickness, Δx1, of an imaging layer, Vpi, may be normal to the hadron beam 1h as shown, e.g., in the examples of FIGS. 6A, 6B, and 9B, such that the hadron beam may be comprised in a single imaging layer. The representation on a same display of the position of the Bragg peak with respect to the position, P1, of the target spot may allow for in situ correction of the initial energy, E1, of the hadron beam to match the positions of the Bragg peak and the target spot.

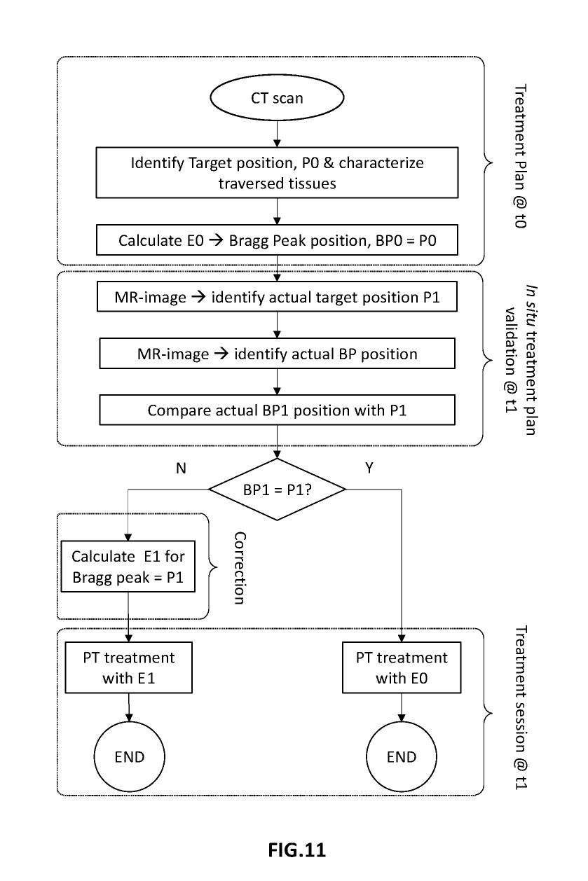

As discussed supra in relation with FIG. 1 (labeled “∃Δ?” →Y therein), absent the positon of the Bragg peak of a hadron beam of initial energy, E0, on the day t1 of a hadron therapy session, if the MR images reveal any change of morphology or position of the tissues surrounding and including the target tissue since the day t0 of the treatment plan, the hadron therapy session generally must be stopped and a new treatment plan established. As illustrated in FIG. 11, by visualizing the hadron beam obtained by embodiments of the present disclosure, a mismatch may be identified between the position of the Bragg peak (BP) and the position, P1, of the target spot (even if P1=P0). Accordingly, embodiments of the present disclosure may allow for correcting in situ the initial energy, E1, such that the Bragg peak falls on the position P1 of the target spot. The correction involves the measurement of the position P1 of the target spot, and of the thicknesses, Lm, with m=40-43 as shown in the example of FIGS. 2A, 2B, and 2C, of the various tissues the hadron beam must traverse to reach the target spot 40s, to determine the distance the hadron beam must travel through the tissues to reach the target spot. Through the determination of the corresponding WEPLs as described above, one may calculate the initial energy, E1, such that the position of the Bragg peak of the hadron beam overlaps with the position, P1, of the target spot 40s. The treatment may thus proceed the same day with the corrected initial energy, E1. This may provide economical benefits as well as improve the health of the patients.

The doses deposited onto the tissues for visualizing the hadron beam path must generally be low, because, in case of a change of morphology of the tissues, a full therapeutic dose reaching healthy tissues may be extremely detrimental to the health of a patient.

Accordingly, the hadron doses deposited for the visualization of the hadron beam may be substantially lower than the therapeutic doses required for treating the target tissue and may have substantially no therapeutic effects. As discussed with respect to FIG. 4C, this may be achieved either by irradiating few target spots, e.g., irradiating 1% to 40% of the target spots of an iso-energy layer, Vti, e.g., 5% to 30% or 10% to 20%. Alternatively or concomitantly, target spots may be irradiated with a hadron beam having an intensity substantially lower than prescribed by the treatment plan. Finally, the irradiation time, ti, may also be reduced, e.g., to the minimum required for acquiring a MR image. In these conditions, the validation of the treatment plan is generally safe for the patient, even if a correction of the initial energy is then required. For example, some embodiments may irradiate only a selection of the target spots 40si,j of the target tissue to yield the relative positions of the Bragg peak, BP1, and the corresponding target spot, 40s, to calculate the initial energy, E1, which may be used during the treatment session to treat all the target spots 40si,j of an iso-energy volume, Vti. The initial energies required for treating target spots, 40(i+1),j, etc., in subsequent iso-energy volumes, Vt(i+1), etc., may either be extrapolated from the initial energy, E1, and/or determined for the iso-energy volume, Vti, or, alternatively or additionally, a selection of target spots 40(i+1),j, etc., of the subsequent energy volumes, Vt(i+1), etc., may be tested as described above.

Embodiments of the present disclosure thus propose solutions for visualizing, by MR imaging the beam path of a hadron beam traversing a tissue. As explained supra, the irradiated excited atoms generally emit, upon relaxation, RF-signals which differ from the RF-signal these same excitable atoms would have emitted were they not irradiated. This phenomenon may provoke a perturbation of the image by attributing an RF-signal to a wrong voxel. If the operator is not aware of the existence of such perturbation, a wrong conclusion may be drawn from such perturbed MR image. It may, therefore, be important to know how to visualize said tissues without artefacts provoked by the irradiated excited atoms. This may be important because comparison between MR images with and without a hadron beam may be required for establishing the trail left by the beam path in case the hadron pulses are synchronized with one or more of the phase gradient step and/or the frequency gradient step. Acquiring the MR-data absent any hadron beam may assist in this regard; however, it may not always be efficient. Accordingly, some embodiments of the present disclosure propose a solution for visualizing tissues as they are being traversed by hadron pulses, without any artefacts provoked by the irradiated excitable atoms A1 on the MR images thus acquired.

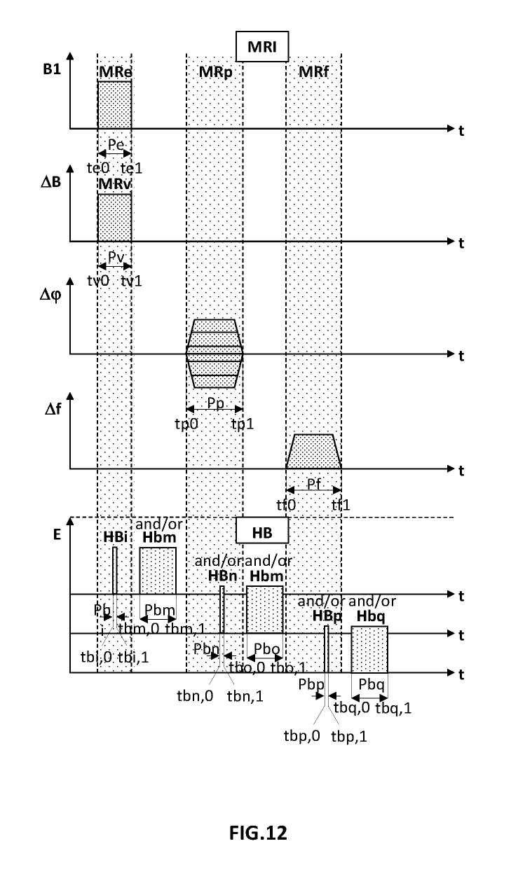

One method of the present disclosure for visualizing an organic body traversed by a hadron beam without artefacts created by said hadron beam is illustrated in the example of FIG. 12. Said method may comprise:
(a) providing a hadron source adapted for directing a hadron beam having an initial energy, E0, along a beam path, Xp, intersecting a target tissue in the organic body;
(b) providing a magnetic resonance imaging device (MRI) for acquiring magnetic resonance data within an imaging volume, Vp, including the target tissue, positioned in a uniform main magnetic field, B0;
(c) acquiring magnetic resonance data from the imaging volume, by applying at least the following steps:
   a layer selection step (MRv) for selecting an imaging layer, Vpi, of the imaging volume, Vp, of thickness, Δxi, measured along the first direction, X1 including creating a magnetic field gradient in a first direction, X1;
   an excitation step (MRe) for exciting the spin of the nuclei of excitable atoms A0, by creating an oscillating electromagnetic field, B1, at a given RF frequency range [fL]i corresponding to the Larmor frequencies of the excitable atoms located within the imaging layer, Vpi, during an excitation period, Pe=(te1-31 te0), wherein te0 and te1 are the times of the beginning and end of the excitation step, respectively;
   a phase gradient step (MRp) for localising along the second direction, X2, the origin of RF signals received by the antennas during relaxation of the excited spins, including creating magnetic field gradients in a second direction, X2, normal to the first direction, X1, (X1⊥X2), during a period Pp=(tp1−tp0), wherein tp0 and tp1 are the times of the beginning and end of the phase gradient step, respectively, with tf0>tp1; and
   a frequency gradient step (MRf)) for localising along the third direction, X3, the origin of RF signals received by the antennas during relaxation of the excited spins including creating magnetic field gradients in a third direction, X3, normal to the first and second directions, (X1⊥X2⊥X3), during a period Pf=(tf1-31 tf0), wherein tf0 and tf1 are the times of the beginning and end of the frequency gradient step, respectively, with tf0>tp1;
(d) directing a hadron beam having the initial energy, E0, along a beam path intersecting said target body in the imaging layer, Vpi, preferably but not necessarily normal to the first direction, X1, in a number, N, of hadron pulses of pulse periods, PBi, wherein, N is an integer greater than 0; and
(e) representing on a display the organic body from the magnetic resonance data acquired by the MRI within the imaging volume, Vp, without interferences from the hadron beam,
   characterized in that, the acquisition of magnetic resonance data and the emission of hadron pulses may be synchronized such that,
   a pulse period PBi, may overlap with an MR-period, Pj, with j=e, f, and/or p, of one of the excitation step, Pe, the phase gradient step, Pp, and the frequency gradient step, Pf, and may be not more than 20% of the MR-period, Pj, it overlaps with, such that PBi≤0.2 Pj, or
   a pulse period PBi, may not overlap with any of the MR-periods, Pj.

Contrary to previously proposed solutions, the representation step (e) of the present method does not require any deletion of the measured values of the MR imaging data samples having been acquired during any MR-period, Pj, overlapping with a pulse period, PBi. Indeed, because there is an overlap of at most 20% between an MR-period, Pj, and a pulse period, PBi, the representation on a display of the organic body may be sufficiently undistorted to yield an MR image representative of the organic body without requiring the deletion of any MR imaging data sample.

Embodiments of the present disclosure also include a medical apparatus for carrying out the foregoing method of visualizing a hadron beam together with the target tissue it must irradiate. The medical apparatus may comprise:
(a) a hadron source adapted for directing a hadron beam 1h having a beam energy, E1, along a beam path in a number, N, of hadron pulses of pulse period, PBi=(tBi,1−tBi,0);
(b) a magnetic resonance imaging device (MRI) 2 for the acquisition of magnetic resonance data from the excitable atoms within an imaging volume, Vp, including the organic body, wherein the MRI comprises:
   a main magnetic unit (2 m) for creating a uniform main magnetic field, B0;
   an RF unit 2e suitable for creating an oscillating electromagnetic field, B1, at a given RF frequency range;
   slice selection coils 2s for creating a magnetic field gradient in a first direction, X1;
   X2-gradient coils 2p for creating magnetic field gradients in a second direction, X2, normal to the first direction, X1, (X1⊥X2);
   X3-gradient coils 2f for creating magnetic field gradients in a third direction, X3, normal to the first and second directions, (X1⊥X2⊥X3); and
   antennas 2a for receiving RF signals emitted by excited atoms upon relaxation;
(c) a controller configured for acquiring magnetic resonance data by implementing the following steps:
   an excitation step (MRe) for exciting the spin of the nuclei of the excitable atoms, comprising creating an electromagnetic field, B1, with the RF unit oscillating at a RF-frequency range, [fL]i, corresponding to the Larmor frequencies of the excitable atoms exposed to an i^{th} magnetic field range, [B0]i=[Bi,0, Bi,1], during an excitation period, Pe=(te1−te0),
   a layer selection step (MRv) applied during the excitation step for selecting a layer, Vpi, of the imaging volume, Vp, of thickness, Δxi, measured along the first direction, X1, by creating a magnetic field gradient along the first direction, X1, of slope dB/dx1=[B0]i/Δxi,
   a phase gradient step (MRp) applied after the excitation and slice selection steps for localising along the second direction, X2, the origin of RF signals received by the antennas by varying a phase of the spins of the nuclei along the second direction, X2, and comprising the step of creating a magnetic field gradient along the second direction, X2, during a period, Pp=(tp1−tp0), and
   a frequency gradient step (MRI) applied after the phase gradient step for localising along the third direction, X3, the origin of RF signals received by the antennas by varying a frequency of the spins of the nuclei along the third direction, X3, and comprising the step of creating a magnetic field gradient along the third direction, X3, during a period Pf=(tf1 tf0); and
(d) a display for representing the organic body from the magnetic resonance data acquired by the MRI within the imaging volume, Vp, as well as for visualizing the beam path,
   characterized in that, the controller may be further configured for synchronizing the acquisition of magnetic resonance data and the emission of hadron pulses, such that an MR-period, Pj, with j=e, f, and/or p, of one or more of the excitation step, Pe, the phase gradient step, Pp, and the frequency gradient step, Pf, may overlap with and not exceed the pulse period, PBi, by more than 10%, Pj≤1.1 PBi, and in that, the MR-period, Pj, may be out of phase with respect to the pulse period PBi by not more than 10%, such that (tBi,0−tj0)/PBi≤0.1, and (tBi,1−01)/PBi≥0.1, with j =e, f, and/or p.

1.-10. (canceled)

11. A computer-implemented method for displaying, on a computer display, a hadron beam traversing an organic body, wherein the hadron beam is provided by a hadron source configured to direct the hadron beam with an initial energy along a beam path intersecting a target tissue in the organic body, the method comprising:
acquiring, from a magnetic resonance imaging device, magnetic resonance data associated with an imaging volume including the target tissue, wherein acquiring the magnetic resonance data from the imaging volume further includes:
selecting an imaging layer of the imaging volume having a first thickness measured along a first direction,
exciting the spin of nuclei of excitable atoms by creating an electromagnetic field oscillating at a given RF frequency range corresponding to Larmor frequencies of the excitable atoms located within the imaging layer during an excitation period,
localizing, along a second direction, an origin of RF signals received by antennas during relaxation of the excited spins, where the second direction is normal to the first direction, during a phase gradient period, and
localizing along a third direction the origin of RF signals received by the antennas during relaxation of the excited spins, where the third direction is normal to the first direction and the second direction, during a frequency gradient period;
directing the hadron beam with the initial energy along the beam path intersecting the target tissue in the imaging layer in one or more hadron pulses having one or more pulse periods;
representing, on a display, the organic body based on the magnetic resonance data;
displaying, on the display, the beam path as a hyposignal, the hyposignal being weaker than the signal generated by a portion of the excitable atoms unexposed to the hadron beam; and
synchronizing the acquisition of the magnetic resonance data and the directing of the hadron beam such that at least one of the excitation period, the phase gradient period, and the frequency gradient period overlaps with and does not exceed one of the pulse periods by more than a first threshold and such that at least one of the excitation period, the phase gradient period, and the frequency gradient period is out of phase with respect to one of the pulse periods by not more than the first threshold.

12. The method of claim 11, wherein the first threshold is 10%.

13. The method of claim 11, wherein the at least one of the excitation period, the phase gradient period, and the frequency gradient period comprises the excitation period, and wherein the excitation period and one of the pulse periods are out of sync by no more than a second threshold.

14. The method of claim 13, wherein the second threshold is 30%.

15. The method of claim 13, wherein the second threshold is 20%.

16. The method of claim 11, wherein one or more of the pulse periods are between 10 ps and 30 ms.

17. The method of claim 16, wherein one or more of the pulse periods are between 5 and 20 ms.

18. The method of claim 11, wherein the one or more hadron pulses comprise at least two pulses, and wherein the two pulses are separated by a separation period.

19. The method of claim 18, wherein the separation period is between 1 ms and 20 ms.

20. The method of claim 11, wherein the excitation period, the phase gradient period, and the frequency gradient period are independently selected from between 1 ms and 100 ms.

21. The method of claim 11, wherein the beam path is substantially normal to the first direction.

22. The method of claim 11, further comprising:
establishing a treatment plan including the initial energy;
comparing, using the display, morphology and thicknesses of tissues traversed by the hadron beam;
displaying, on the display, the position of a Bragg peak of the hadron beam; and
when the position of the Bragg peak and a position of the target tissue differ by more than a second threshold, correcting the initial energy such that the position of the Bragg peak and the position of the target tissue are within the second threshold.

23. The method of claim 11, wherein the imaging volume is controlled by generating a magnetic gradient along at least one of the first direction, the second direction, and the third direction to control a thickness of the imaging volume along the first direction, the second direction, or the third direction.

24. A computer-implemented method for displaying, on a computer display, an organic body traversed by a hadron beam, wherein the hadron beam is provided by a hadron source configured to direct the hadron beam with an initial energy along a beam path intersecting a target tissue in the organic body, the method comprising:
acquiring, from a magnetic resonance imaging device, magnetic resonance data within an imaging volume including the target tissue and positioned in a uniform main magnetic field;
acquiring the magnetic resonance data from the imaging volume, wherein acquiring the magnetic resonance data from the imaging volume further includes:
selecting an imaging layer of the imaging volume having a first thickness measured along a first direction,
exciting the spin of nuclei of excitable atoms by creating an electromagnetic field oscillating at a given RF frequency range corresponding to Larmor frequencies of the excitable atoms located within the imaging layer during an excitation period,
localizing, along a second direction and during a phase gradient period, the origin of RF signals received by antennas during relaxation of the excited spins, where the second direction is normal to the first direction, and
localizing, along a third direction and during a frequency gradient period, the origin of RF signals received by the antennas during relaxation of the excited spins, where the third direction is normal to the first direction and the second direction;
directing the hadron beam with the initial energy along the beam path intersecting the target tissue in the imaging layer in one or more hadron pulses having one or more pulse periods;
displaying, on the display, the organic body based on the magnetic resonance data; and
synchronizing the acquisition of the magnetic resonance data and the directing of the hadron pulses such that one or the pulse periods either overlaps with and has a length not exceeding a fraction of at least one of the excitation period, the phase gradient period, and the frequency gradient period or does not overlap with at least one of the excitation period, the phase gradient period, and the frequency gradient period.

25. The method of claim 24, wherein the fraction comprises 20%.

26. A medical apparatus, comprising:
a hadron source for directing a hadron beam with an initial energy along a beam path in one or more hadron pulses having one or more pulse periods, the beam path intersection an organic body having excitable atoms;
a magnetic resonance imaging device for acquiring magnetic resonance data from a portion of the excitable atoms within an imaging volume including the organic body, the magnetic resonance imaging device including:
a main magnetic unit for generating a uniform main magnetic field,
an RF unit for generating an electromagnetic field oscillating at a given RF frequency range,
one or more first selection coils for generating a magnetic field gradient in a first direction,
one or more first gradient coils for generating magnetic field gradients in a second direction normal to the first direction,
one or more second gradient coils for generating magnetic field gradients in a third direction normal to the first direction and the second direction, and
one or more antennas for receiving RF signals emitted by excited atoms upon relaxation;
a controller configured to acquire the magnetic resonance data by:
exciting the spin of nuclei of the excitable atoms using the one or more first gradient coils and the one or more second gradient coils during an excitation period,
selecting an imaging layer of the imaging volume having a first thickness measured along the first direction during the excitation period,
localizing, along the second direction, the origin of RF signals received by the antennas during a phase gradient period,
localizing, along the third direction, the origin of RF signals received by the antennas during a frequency gradient period; and
a display for representing the organic body based on the magnetic resonance data and for visualizing the beam path,
wherein the controller is further configured to synchronize the acquisition of the magnetic resonance data with the directing of the hadron pulses such that at least one of the excitation period, the phase gradient period, and the frequency gradient period overlaps with and does not exceed one of the pulse periods by more than a threshold and such that at least one of the excitation period, the phase gradient period, and the frequency gradient period is out of phase with respect to one of the pulse periods by not more than the threshold.

27. The medical apparatus of claim 26, wherein the threshold is 10%.

28. The medical apparatus of claim 26, wherein the controller is further configured to synchronize the acquisition of the magnetic resonance data with the directing of the hadron pulses such that the excitation period and one of the pulse periods are out of sync by no more than a second threshold.

29. The medical apparatus of claim 28, wherein the second threshold is 30%.

30. The medical apparatus of claim 28, wherein the second threshold is 20%.
